SEQUENCE LISTING (12 sequences)

SEQ ID NO: 1 - DNA, 4477 bp, Homo Sapiens
  CDS (90)...(2309)
gcggccgccgcggccaccgcccactcggggctggccagcggcgggcggccggggcgcagagaacggcctggctgggcgagcgcacggccatggccccgtggctgcagctctgctccgtcttctttacggtcaacgcctgcctcaacggctcgcagctggctgtggccgctggcgggtccggccgcgcgcggggcgccgacacctgtggctggaggggagtggggccagccagcagaaacagtgggctgtacaacatcaccttcaaatatgacaattgtaccacctacttgaatccagtggggaagcatgtgattgctgacgcccagaatatcaccatcagccagtatgcttgccatgaccaagtggcagtcaccattctttggtccccaggggccctcggcatcgaattcctgaaaggatttcgggtaatactggaggagctgaagtcggagggaagacagtgccaacaactgattctaaaggatccgaagcagctcaacagtagcttcaaaagaactggaatggaatctcaacctttcctgaatatgaaatttgaaacggattatttcgtaaaggttgtcccttttccttccattaaaaacgaaagcaattaccaccctttcttctttagaacccgagcctgtgacctgttgttacagccggacaatctagcttgtaaacccttctggaagcctcggaacctgaacatcagccagcatggctcggacatgcaggtgtccttcgaccacgcaccgcacaacttcggcttccgtttcttctatcttcactacaagctcaagcacgaaggacctttcaagcgaaagacctgtgagcaggagcaaactacagagatgaccagctgcctccttcaaaatgtttctccaggggattatataattgagctggtggatgacactaacacaacaagaaaagtgatgcattatgccttaaagccagtgcactccccgtgggccgggcccatcagagccgtggccatcacagtgccactggtagtcatatcggcattcgcgacgctcttcactgtgatgtgccgcaagaagcaacaagaaaatatatattcacatttagatgaagagagctctgagtcttccacatacactgcagcactcccaagagagaggctccggccgcggccgaaggtctttctctgctattccagtaaagatggccagaatcacatgaatgtcgtccagtgtttcgcctacttcctccaggacttctgtggctgtgaggtggctctggacctgtgggaagacttcagcctctgtagagaagggcagagagaatgggtcatccagaagatccacgagtcccagttcatcattgtggtttgttccaaaggtatgaagtactttgtggacaagaagaactacaaacacaaaggaggtggccgaggctcggggaaaggagagctcttcctggtggcggtgtcagccattgccgaaaagctccgccaggccaagcagagttcgtccgcggcgctcagcaagtttatcgccgtctactttgattattcctgcgagggagacgtccccggtatcctagacctgagtaccaagtacagactcatggacaatcttcctcagctctgttcccacctgcactcccgagaccacggcctccaggagccggggcagcacacgcgacagggcagcagaaggaactacttccggagcaagtcaggccggtccctatacgtcgccatttgcaacatgcaccagtttattgacgaggagcccgactggttcgaaaagcagttcgttcccttccatcctcctccactgcgctaccgggagccagtcttggagaaatttgattcgggcttggttttaaatgatgtcatgtgcaaaccagggcctgagagtgacttctgcctaaaggtagaggcggctgttcttggggcaaccggaccagccgactcccagcacgagagtcagcatgggggcctggaccaagacggggaggcccggcctgcccttgacggtagcgccgccctgcaacccctgctgcacacggtgaaagccggcagcccctcggacatgccgcgggactcaggcatctatgactcgtctgtgccctcatccgagctgtctctgccactgatggaaggactctcgacggaccagacagaaacgtcttccctgacggagagcgtgtcctcctcttcaggcctgggtgaggaggaacctcctgcccttccttccaagctcctctcttctgggtcatgcaaagcagatcttggttgccgcagctacactgatgaactccacgcggtcgcccctttgtaacaaaacgaaagagtctaagcattgccactttagctgctgcctccctctgattccccagctcatctccctggttgcatggcccacttggagctgaggtctcatacaaggatatttggagtgaaatgctggccagtacttgttctcccttgccccaaccctttaccggatatcttgacaaactctccaattttctaaaatgatatggagctctgaaaggcatgtccataaggtctgacaacagcttgccaaatttggttagtccttggatcagagcctgttgtgggaggtagggaggaaatatgtaaagaaaaacaggaagatacctgcactaatcattcagacttcattgagctctgcaaactttgcctgtttgctattggctaccttgatttgaaatgctttgtgaaaaaaggcacttttaacatcatagccacagaaatcaagtgccagtctatctggaatccatgttgtattgcagataatgttctcatttatttttgatgtagaatttacattgccatgggtgttaaataagctttgagtcaaaagtcaagaaagtgactgaatatacagtcaccttttatgaaatgagtctctgtgttactgggtggcatgactgattgaggtgaagctcacggggccaggctgaccgtcttgaccgttccacttgagataggttggtcatcgtgcagaaggccccaggacctcagcacacacagcctcctcttggtctgagtaggcatcatgtgggggccagatctgcctgctgtttccatgggttacatttactgtgctgtatctcagatgttggtgtctggaagtttattcttaagagactgctacccagctggtctgtattattggaagttgcagttcgtgctttggttggccttctggtctaaagctgtgtcctgaatattagggatcacaattcactgaaatacagcagtgtgtggaggtgatggccagttaatctgctgaactggttttgactaatgacaaacctctttttaagatggtagaatggaggtgatagtcacaaaagtaaatgttccatttttatgaatgactttctacagagtttctatttctaaagaaaaaacaattgttcacatcccatctgatgattagcatgtgtgtaatgaatgctgtcttggtctcccctgtggaaacccttctccctgtgccttagagcaggtgtgtacatctctcactacctttctcatgggtgctgttagattttggcacccgttttctcagcattcagcccagggaatgtggttttcacttcttcgtcagataagaccaacatgaaggggtatgttgagaaacatcctgaggcaaggtgggaggtgggatggggcaggactttcccttccaagcacatgcatggcaggtggggaaaggggggcttgcacccctgctggaaagaaaaggtttgtgtatatttctgatgcaaatgtcatactcactgctctgtaaaggcagctggcagctttttgggaaaagaacgtgctcgtctgttctctggcatcaagtttcttgcagctgctctgagggagagacagtgagctgcaagactgcctccccataacaacaggcaactcagagaagagtcattttatgttgttcctatggaatctggaatgagtgcagagctcctacccacacatgactgccccgccatttcatcctaggcattctgtgaaggagattggttagtccaaacttgctaacatacgaaaattcacttggaacatgatgagagatttcttattgaggccaagagatgtttcctgtcccagaggaaccattaggagtcgcttttagggtattcagctttgttcatgaaataaggcatctctgagaaagtggccccagggagagaatggaggactgggaggagaagcattaactgagctccaagggtgtgtgggcagagagcttgctatgtgaactcactccttaagaaaatggaagagaaaaagagagtgctagttaaaaaatcgggatgttttagtttggatttagggttttgatacttatgttgaaatactaatgtttctgatcaataaaatcaaactcttaatataccgagtaatgaaaccatagtgtgattgcctcagaataaattgagaagtccaaaaaaaaaaaaaaaaaaaaaaaaa

SEQ ID NO: 2 - protein, 739 aa, Homo Sapiens [sequence omitted]

SEQ ID NO: 3 - DNA, 3083 bp, Homo Sapiens
  CDS (22)...(2241)
tggctgggcgagcgcacggccatggccccgtggctgcagctctgctccgtcttctttacggtcaacgcctgcctcaacggctcgcagctggctgtggccgctggcgggtccggccgcgcgtggggcgtcgacacctgtggctggaggggagtggggccagccagcagaaacagtgggctgtacaacatcaccttcaaatatgacaattgtaccacctacttgaatccagtggggaagcatgtgattgctgacgcccagaatatcaccatcagccagtatgcttgccatgaccaagtggcagtcaccattctttggtccccaggggccctcggcatcgaattcctgaaaggatttcgggtaatactggaggagctgaagtcggagggaagacagtgccaacaactgattctaaaggatccgaagcagctcaacagtagcttcaaaagaactggaatggaatctcaacctttcctgaatatgaaatttgaaacggattatttcgtaaaggttgtcccttttccttccattaaaaacgaaagcaattaccaccctttcttctttagaacccgagcctgtgacctgttgttacagccggacaatctagcttgtaaacccttctggaagcctcggaacctgaacatcagccagcatggctcggacatgcaggtgtccttcgaccacgcaccgcacaacttcggcttccgtttcttctatcttcactacaagctcaagcacgaaggacctttcaagcgaaagacctgtaagcaggagcaaactacagagacgaccagctgcctccttcaaaatgtttctccaggggattatataattgagctggtggatgacactaacacaacaagaaaagtgatgcattatgccttaaagccagtgcactccccgtgggccgggcccatcagagccatggccatcacagtgccactggtagtcatatcggcattcgcgacgctcttcactgtgatgtgccgcaagaagcaacaagaaaatatatattcacatttagatgaagagagctctgagtcttccacatacactgcagcactcccaagagagaggctccggccgcggccgaaggtctttctctgctattccagtaaagatggccagaatcacatgaatgtcgtccagtgtttcgcctacttcctccaggacttctgtggctgtgaggtggctctggacctgtgggaagacttcagcctctgtagagaagggcagagagaatgggtcatccagaagatccacgagtcccagttcatcattgtggtttgttccaaaggtatgaagtactttgtggacaagaagaactacaaacacaaaggaggtggccgaggctcggggaaaggagagctcttcctggtggcggtgtcagccattgccgaaaagctccgccaggccaagcagagttcgtccgcggcgctcagcaagtttatcgccgtctactttgattattcctgcgagggagacgtccccggtatcctagacctgagtaccaagtacagactcatggacaatcttcctcagctctgttcccacctgcactcccgagaccacggcctccaggagccggggcagcacacgcgacagggcagcagaaggaactacttccggagcaagtcaggccggtccctatacgtcgccatttgcaacatgcaccagtttattgacgaggagcccgactggttcgaaaagcagttcgttcccttccatcctcctccactgcgctaccgggagccagtcttggagaaatttgattcgggcttggttttaaatgatgtcatgtgcaaaccagggcctgagagtgacttctgcctaaaggtagaggcgcctgttcttggggcaaccggaccagccgactcccagcacgagagtcagcatgggggcctggaccaagacggggaggcccggcctgcccttgacggtagcgccgccctgcaacccctgctgcacacggtgaaagccggcagcccctcggacatgccgcgggactcaggcatctatgactcgtctgtgccctcatccgagctgtctctgccactgatggaaggactctcgacggaccagacagaaacgtcttccctgacggagagcgtgtcctcctcttcaggcctgggtgaggaggaacctcctgcccttccttccaagctcctctcttctgggtcatgcaaagcagatcttggttgccgcagctacactgatgaactccacgcggtcgcccctttgtaacaaaacgaaagagtctaagcattgccactttagctgctgcctccctctgattccccagctcatctccctggttgcatggcccacttggagctgaggtctcatacaaggatatttggagtgaaatgctggccagtacttgttctcccttgccccaaccctttaccggatatcttgacaaactctccaattttctaaaatgatatggagctctgaaaggcatgtccataaggtctgacaacagcttgccaaatttggttagtccttggatcagagcctgttgtgggaggtagggaggaaatatgtaaagaaaaacaggaagatacctgcactaatcattcagacttcattgagctctgcaaactttgcctgtttgctattggctaccttgatttgaaatgctttgtgaaaaaaggcacttttaacatcatagccacagaaatcaagtgccagtctatctggaatccatgttgtattgcagataatgttctcatttatttttgatgtagaatttacattgccatgggtgttaaataagctttgagtcaaaagtcaagaaagtgactgaatatacagtcaccttttatgaaatgaaaaaaaaaaaaaaaaaaaaaaaaaaagggcggccgctctagagtatccctcgaggggcccaagcttacgcgtacccagctttcttgtacaaagtggtccctatagtgagtcgtattataagctaggcactggccgtcgttttacaacgtcgtgactgggaaaactgctagcttgggatctttgtgaaggaaccttacttctgtggtgtgacataattggacaaactacctacagagatttaaagctctaag

SEQ ID NO: 4 - protein, 739 aa, Homo Sapiens
MAPWLQLCSVFFTVNACLNGSQLAVAAGGSGRAWGVDTCGWRGVGPASRNSGLYNITFKYDNCTTYLNPVGKHVIADAQNITISQYACHDQVAVTILWSPGALGIEFLKGFRVILEELKSEGRQCQQLILKDPKQLNSSFKRTGMESQPFLNMKFETDYFVKVVPFPSIKNESNYHPFFFRTRACDLLLQPDNLACKPFWKPRNLNISQHGSDMQVSFDHAPHNFGFRFFYLHYKLKHEGPFKRKTCKQEQTTETTSCLLQNVSPGDYIIELVDDTNTTRKVMHYALKPVHSPWAGPIRAMAITVPLVVISAFATLFTVMCRKKQQENIYSHLDEESSESSTYTAALPRERLRPRPKVFLCYSSKDGQNHMNVVQCFAYFLQDFCGCEVALDLWEDFSLCREGQREWVIQKIHESQFIIVVCSKGMKYFVDKKNYKHKGGGRGSGKGELFLVAVSAIAEKLRQAKQSSSAALSKFIAVYFDYSCEGDVPGILDLSTKYRLMDNLPQLCSHLHSRDHGLQEPGQHTRQGSRRNYFRSKSGRSLYVAICNMHQFIDEEPDWFEKQFVPFHPPPLRYREPVLEKFDSGLVLNDVMCKPGPESDFCLKVEAPVLGATGPADSQHESQHGGLDQDGEARPALDGSAALQPLLHTVKAGSPSDMPRDSGIYDSSVPSSELSLPLMEGLSTDQTETSSLTESVSSSSGLGEEEPPALPSKLLSSGSCKADLGCRSYTDELHAVAPL

SEQ ID NO: 5 - DNA, 28 bp, Artificial Sequence
  Oligonucleotide primer
ccgaagcttgtcgacacctgtggctgga

SEQ ID NO: 6 - DNA, 29 bp, Artificial Sequence
  Oligonucleotide primer
ccgtctagattacaaaggggcgaccgcgt

SEQ ID NO: 7 - DNA, 28 bp, Artificial Sequence
  Oligonucleotide primer
ccgaagcttgtcgacacctgtggctgga

SEQ ID NO: 8 - DNA, 34 bp, Artificial Sequence
  Oligonucleotide primer
ccgtctagattaccggagcctctctcttgggagt

SEQ ID NO: 9 - DNA, 34 bp, Artificial Sequence
  Oligonucleotide primer
ccgaagcttcattatgccttaaagccagtgcact

SEQ ID NO: 10 - DNA, 29 bp, Artificial Sequence
  Oligonucleotide primer
ccgtctagattacaaaggggcgaccgcgt

SEQ ID NO: 11 - DNA, 28 bp, Artificial Sequence
  Oligonucleotide primer
ccgaagcttgtcgacacctgtggctgga

SEQ ID NO: 12 - DNA, 36 bp, Artificial Sequence
  Oligonucleotide primer
tgctctagattagggaacgaactgcttttcgaacca

(19) United States
(12) Patent Application Publication
(10) Pub. No.: US 2004/0235104 A1
(43) Pub. Date: Nov. 25, 2004

(54) Human SEF molecule and uses therefor

(75) Inventor: Ruey-Bing Yang, Taipei (TW)

Correspondence Address: Tracy M. Sioussat, Millennium Pharmaceuticals, Inc., 40 Landsdowne Street, Cambridge, MA, 02139, US

(73) Assignee: Millennium Pharmaceuticals, Inc.

(21) Appl. No.: 10/842,006

(22) Filed: May 7, 2004

Related U.S. Application Data
(60) Provisional application No. 60/469,522, filed on May 8, 2003.

(51) Int. Cl. C12N 9/20; C07H 21/04; C07K 16/40

(52) U.S. Cl. 435/69.1; 435/198; 435/320.1; 435/325; 530/388.26; 536/23.2

(57) ABSTRACT

The invention provides isolated nucleic acids molecules, designated SEF nucleic acid molecules. The invention also provides antisense nucleic acid molecules, recombinant expression vectors containing SEF nucleic acid molecules, host cells into which the expression vectors have been introduced, and nonhuman transgenic animals in which a SEF gene has been introduced or disrupted. The invention still further provides isolated SEF proteins, fusion proteins, antigenic peptides and anti-SEF antibodies. Diagnostic and therapeutic methods utilizing compositions of the invention are also provided.

### CROSS REFERENCE TO RELATED APPLICATIONS

This application claims the benefit of U.S. Provisional Application No. 60/469,522, filed May 8, 2003, the contents of which are incorporated herein by this reference in their entirety.

### BACKGROUND OF THE INVENTION

Receptor tyrosine kinases (RTKs) play an important role in many cellular processes. RTKs have an extracellular ligand binding domain and an intracellular tyrosine kinase domain that is activated after ligand binding and receptor dimerization. Ulrich, A., et al., Cell, 61:203 (1990). This signaling event triggers a cascade resulting in the modulation of may cellular events determining proliferation, differentiation and morphogenesis in positive and negative ways. Many different types of disorders, e.g., cancer, are triggered upon disturbances in this pathway. Gene mutations giving rise to altered protein products have been shown to alter the regulatory mechanism influencing cellular proliferation, resulting in tumor initiation and progression. Shawver, et al., Receptor Tyrosine Kinases as Targets for Inhibition of Angiogenesis, DDT (Elsevier Science Ltd.), 2(2):50 (1997).

Fibroblast Growth Factors (FGFs) are members of a family of polypeptides that regulate a RTK known as the Fibroblast Growth Factor Receptor (FGFR), Fibroblast Growth Factors (FGFs) are potent regulators of a variety of cellular processes including proliferation, differentiation, migration, and morphogenesis (Burgess, W. H. and Maciag, T (1989) Annu. Rev. Biochem. 58: 575-606; Rifkin, D. B. and Moscatelli, D. (1989) J. Cell Biol. 109:1-6). These proteins play important roles in normal development (Yamaguchi, J. P. and Rossant, J. (1995) Curr. Opin. Genet. Dev. 485-491; Kimmelman et al. (1988) Science 242:1053-1056; Slack et al. (1988) Development 103: 581-590), in the maintenance of tissue, and in wound healing and repair (Clarke et al. (1993) J. Cell Sci.106: 121-133; Cuevas et al. (1988) Biochem. Biophys. Res. Commun. 156: 611-618). FGF family members have also been implicated in a wide range of pathological conditions, including tumorigenesis and metastasis (Davies et al. (1996) Int. J. Cancer 65: 104-111; Myoken et al. (1996) Int. J. Cancer 65: 650-657).

Target cell responses are mediated, in part, by the binding of FGF ligands to cognate FGF receptors (FGFR) that possess intrinsic tyrosine kinase activity. There are currently four known genes encoding FGF receptors (FGFR-1, FGFR-2, FGFR-3, and FGFR-4), which can give rise to a variety of protein isoforms via alternative RNA splicing (Galzie, Z. et al. (1997) Biochem. Cell. Biol., 75:669-685).

The cellular effects mediated by tyrosine kinases and the signaling molecules that associate with them, make attractive targets for the development of new therapeutic molecules. For example, over expression of tyrosine kinases, such as HER2, can play a role in the development of cancer and that antibodies capable of blocking the activity of this enzyme can abrogate tumor growth. Slamon et al. Science, 235:177 (1987).

### SUMMARY OF THE INVENTION

The invention relates to SEF, novel sef alleles (e.g., SEQ ID NO:1), antibodies and antigen-binding fragments, isolated nucleic acids, recombinant constructs and vectors, host cells, methods of production, screening assays and diagnostic, prognostic and therapeutic methods described herein.

In one aspect, the invention relates to an isolated and/or recombinant nucleic acid that encodes a SEF protein or fragment as described herein. The isolated and/or recombinant nucleic acid can comprise a nucleotide sequence having at least about 90% nucleotide sequence identity with SEQ ID NO: 1 or the coding sequence thereof, and contain one or more of the following: a cytosine at a position corresponding to position 100 of SEQ ID NO:1, a cytosine at a position corresponding to position 107 of SEQ ID NO:1, a guanine at a position corresponding to position 742 of SEQ ID NO:1, a thymine at a position corresponding to position 764 of SEQ ID NO:1, a guanine at a position corresponding to position 901 of SEQ ID NO:1, and a guanine at a position corresponding to position 1822 of SEQ ID NO:1.

The isolated and/or recombinant nucleic acid can encode a SEF protein (e.g., a human SEF). In certain embodiments the isolated and/or recombinant nucleic acid encodes a SEF allelic variant, wherein said SEF allelic variant comprises an amino acid sequence that has at least about 90% amino acid sequence identity with SEQ ID NO:2 and comprises one or more of the following: an arginine at a position corresponding to position 34 of SEQ ID NO:2, an alanine at a position corresponding to position 36 of SEQ ID NO:2, a glutamic acid at a position corresponding to position 248 of SEQ ID NO:2, a methionine at a position corresponding to position 255 of SEQ ID NO:2, a valine at a position corresponding to position 301 of SEQ ID NO:2, and an alanine at a position corresponding to position 608 of SEQ ID NO:2.

In another aspect, the invention relates to an isolated SEF protein (e.g., mature SEF). In some embodiments, the isolated SEF protein has at least about 90% amino acid sequence identity with SEQ ID NO:2 and comprises one or more of the following: an arginine at a position corresponding to position 34 of SEQ ID NO:2, an alanine at a position corresponding to position 36 of SEQ ID NO:2, a glutamic acid at a position corresponding to position 248 of SEQ ID NO:2, a methionine at a position corresponding to position 255 of SEQ ID NO:2, a valine at a position corresponding to position 301 of SEQ ID NO:2, and an alanine at a position corresponding to position 608 of SEQ ID NO:2. The invention also relates to fragment of a SEF protein comprising an amino acid sequence selected from the group consisting of amino acid residues 36-739 of SEQ ID NO:2, amino acid residues 284-739 of SEQ ID NO:2, amino acid residues 36-353 of SEQ ID NO:2, and amino acid residues 36-510 of SEQ ID NO:2. The invention also relates to a fusion protein comprising a SEF protein or fragment as described herein and a second moiety operably linked thereto.

The invention also relates to an isolated and/or recombinant nucleic acid encoding a fusion protein as described herein, and further relates to isolated and/or recombinant constructs and vectors that comprise a recombinant nucleic acid of the invention.

The invention also relates to a host cell comprising a recombinant nucleic acid, recombinant construct or vector of the invention. In certain embodiments, the host cell comprises a recombinant nucleic acid that encodes SEF or a SEF fragment, and is operably linked to an expression control element.

The invention also relates to methods of producing a SEF protein or SEF fragment. In certain embodiments, the method comprises maintaining a host cell comprising a recombinant nucleic acid that encodes a SEF protein or a SEF fragment under conditions suitable for expression of the nucleic acid. The SEF protein or SEF fragment can be further isolated or purified.

The invention also relates to an antibody or an antigen-binding fragment thereof that binds a SEF protein or fragment as described herein. In one embodiment, the antibody or antigen-binding fragment has binding specificity for a polypeptide comprising SEQ ID NO:2, but does not have binding specificity for a polypeptide of SEQ ID NO:4.

The present invention also provides a method for detecting the presence of a SEF allele in a sample. In certain embodiments, the method comprises combining a sample with a nucleic acid probe which selectively hybridizes to the nucleic acid sequence of SEQ ID NO:1 under high stringency conditions; and detecting whether the nucleic acid probe hybridizes to a nucleic acid in the sample, wherein hybridization is indicative of the presence of a SEF allele.

The invention further provides a method for detecting the presence of a SEF polypeptide. In certain embodiments, the method comprises combining a sample with an antibody or antigen-binding fragment thereof that specifically binds a polypeptide having the amino acid sequence of SEQ ID NO:2, and does not bind SEQ ID NO:4, where formation of a SEF/antibody or SEF/antigen-binding complex is indicative that a SEF polypeptide is present in the sample.

The invention also provides a kit comprising a probe which selectively hybridizes to the nucleic acid sequence of SEQ ID NO:1, under high stringency conditions and instructions for use. In another aspect, the invention provides a kit comprising an antibody or antigen-binding fragment thereof which selectively binds to a SEF polypeptide amino acid sequence of SEQ ID NO:2 and instructions for use. In a particular embodiment, the antibody or antigen-binding fragment does not have binding specificity for a polypeptide of SEQ ID NO:4.

In another aspect, the invention provides for a method for identifying an agent which binds and/or modulates function of a SEF polypeptide as described herein.

The invention further provides a method of modulating the activity of a SEF polypeptide (e.g., a polypeptide comprising the amino acid sequence of SEQ ID NO:2) by contacting the polypeptide with a compound which modulates the activity of SEQ ID NO:2.

The invention also provides for a method of identifying an individual that has a SEF mediated disorder or is at risk of developing a SEF mediated disorder, comprising detecting or measuring expression of a SEF allele in said individual or in a sample obtained from said individual, and comparing the detected or measured expression with a suitable control. Increased or decreased expression relative to said control is indicative that the individual has or is at risk of developing a SEF mediated disorder. In certain embodiments, the SEF allele encodes a polypeptide having at least about 90% amino acid sequence identity with SEQ ID NO:2 and comprises an arginine at a position corresponding to position 34 of SEQ ID NO:2, an alanine at a position corresponding to position 36 of SEQ ID NO:2, a glutamic acid at a position corresponding to position 248 of SEQ ID NO:2, a methionine at a position corresponding to position 255 of SEQ ID NO:2, a valine at a position corresponding to position 301 of SEQ ID NO:2, and/or an alanine at a position corresponding to position 608 of SEQ ID NO:2.

In a particular aspect, the invention relates to SEF and cancer. In particular embodiments, SEF expression serves as a biological marker for cancer (e.g., breast cancer). Accordingly, cancer can be diagnosed, graded and efficacy of therapy can be evaluated by detecting or measuring expression of SEF in a tumor. In other embodiments, SEF protein (e.g., SEQ ID NO:2) is a therapeutic target for tumor therapy (e.g., breast tumor).

### BRIEF DESCRIPTION OF THE DRAWING

FIG. 1 depicts SEF dimer formation. The experiment is described in Example 6. Briefly, three separate transfections are depicted at the top of FIG. 1. SEF constructs are listed at the top of the Figure as V (vector) Flag_SEF, and SEF_Myc. Plus signs indicate constructs that were used in the transfection. IP (immunoprecipitation) lists the antibody used in the IP, while WB (western blot) lists the antibody used to detect the blot.

### DETAILED DESCRIPTION OF THE INVENTION

As described herein, a nucleic acid encoding a novel IL-17 like receptor, SEF (Similar Expression of FGF genes), was cloned from a human cDNA library. Sequence analysis of the clone revealed an open reading frame of 2220 nucleotides (nucleotides 90 to 2309 of SEQ ID NO:1), encoding a protein of 739 amino acids (SEQ ID NO:2). The mature SEF protein, amino acids 36 to 739 of SEQ ID NO:2, contains an extracellular domain from about amino acid residues 36 to 283, a transmembrane region from about amino acid residues 284 to 305, an intracellular region from about amino acid residues 306 to 739, and a SH3 interacting region from about amino acid residues 511 to 739 of SEQ ID NO:2.

Expression studies indicate that SEF is expressed in many different tissue and cell types (Example 2) including HUVECs and various tumors. Differential SEF expression was observed in breast tumors, breast tumor cell lines (Example 9) tumor hemangioma tissue, prostate tumor tissue, and colon tumor tissue (Example 10)

SEF functional studies indicate that SEF is involved in regulating the FGF induced signaling response. Results indicate SEF inhibits MAPK phosphorylation (Example 4). Furthermore various SEF domain constructs demonstrated that the intracellular and putative SH3 binding domains were significant for inhibition of FGF signaling. Confirmatory studies indicate that the inhibitory effect of SEF on FGF signaling effects downstream promoter elements, e.g., FiRE activation (Example 7). Additionally the inventors of the present invention discovered that SEF forms a complex with the FGF receptor (Example 5).

The results of the studies described herein indicate that SEF is involved in FGF induced signaling, cellular proliferation, and angiogenesis. Differential SEF expression in ovarian, breast, hamanginomas, prostate and colon cancers are useful in the diagnosis and treatment of these disorders. The results further indicate that SEF can be used to detect the presence of cancer, e.g., hemangiomas, breast cancer, ovarian cancer, prostate cancer, and colon cancer and that SEF and agents that modulate activity of SEF can be administered to treat cancer. Methods of treating cancer are also provided.

Polypeptides and Proteins

The present invention relates to isolated and/or recombinant (including, e.g., essentially pure) proteins or polypeptides designated mammalian SEF proteins and variants thereof. In a preferred embodiment, the isolated and/or recombinant proteins of the present invention comprise the amino acid sequence of SEQ ID NO:2. In another embodiment, the isolated and/or recombinant proteins of the invention comprise fragments of SEQ ID NO:2 that contain one or more of, arginine 34 of SEQ ID NO:2, alanine 36 of SEQ ID NO:2, glutamic acid 248 of SEQ ID NO:2, methionine 255 of SEQ ID NO:2, valine 301 of SEQ ID NO:2, and alanine 608 of SEQ ID NO:2. In other embodiments the isolated and/or recombinant proteins or polypeptides designate mammalian SEF proteins and variants there of with at least one property, activity or function characteristic of a mammalian SEF protein (as defined herein) such as a binding activity (e.g., FGF receptor binding activity), or a receptor tyrosine kinase regulatory function (e.g., inhibition of FGFR signaling). The SEF regulatory function can be measured directly or indirectly by measuring receptor signaling activity (e.g., inhibition of FGF signaling, inhibition of MAPK phosphorylation, and inhibition of FiRE activation) and, cellular response function (e.g., inhibition of proliferation, migration, angiogenesis). For example, as shown herein, a human SEF protein (SEQ ID NO:2), can inhibit FGF induced signaling and resulting cellular responses (e.g., proliferation). In one embodiment proteins of the present invention can inhibit FGF induced responses, e.g., proliferation, migration, differentiation and embryonic patterning (Boilly et al. (2000) Cytokine Growth Factor Reviews: 11(4):295; Kato et al. (1999) Cellular and Molecular Biology. 45(5):631).

Proteins or polypeptides referred to herein as “isolated” are proteins or polypeptides purified to a state beyond that in which they exist in mammalian cells, and include proteins or polypeptides obtained by methods described herein, similar methods or other suitable methods, including essentially pure proteins or polypeptides, proteins or polypeptides produced by chemical synthesis (e.g., synthetic peptides), by combinations of biological and chemical methods, and by recombinant methods. The proteins can be obtained in an isolated state (e.g., in a powder or other composition) of at least about 50% homogeneity, preferably at least about 75% homogeneity, more preferably at least about 90% homogeneity, or in essentially pure form. Proteins or polypeptides referred to herein as “recombinant” are proteins or polypeptides produced by the expression of recombinant nucleic acids.

As used herein “mammalian SEF protein” refers to naturally occurring or endogenous mammalian SEF proteins and to proteins having an amino acid sequence which is the same as that of a naturally occurring or endogenous corresponding mammalian SEF protein (e.g., recombinant proteins). Accordingly, as defined herein, the term includes mammalian SEF protein, including mature protein, polymorphic or allelic variants, and other isoforms of mammalian SEF (e.g., produced by alternative splicing or other cellular processes), and modified or unmodified forms of the foregoing (e.g., glycosylated, unglycosylated, phosphorylated or unphosphorylated SEF proteins). Naturally occurring or endogenous mammalian SEF proteins include wild type or mature proteins, such as mature SEF or allelic variants and other isoforms which occur naturally in mammals (e.g., humans, non-human primates). Such proteins can be recovered from a source which naturally produces mammalian SEF, for example endothelial cells. These proteins and mammalian SEF proteins having the same amino acid sequence as a naturally occurring or endogenous corresponding mammalian SEF, are referred to by the name of the corresponding mammal. For example, where the corresponding mammal is a human, the protein is designated as a human SEF protein (e.g., a recombinant human SEF produced in a suitable host cell).

As used herein, “biologically active variants” of mammalian SEF proteins include biologically active fragments, biologically active mutant proteins, and/or biologically active fusion proteins (e.g., produced via mutagenesis and/or recombinant techniques, e.g., SEF fusion proteins described in Examples 4 to 7). Generally, fragments or portions of mammalian SEF proteins encompassed by the present invention include those having a deletion (e.g., one or more deletions) of an amino acid (e.g., one or more amino acids) relative to the mature mammalian SEF protein (such as N-terminal, C-terminal or internal deletions). In a one embodiment at least one, two, three, four, five, six, seven, eight, nine, or ten amino acids are deleted. Fragments or portions in which only contiguous amino acids have been deleted or in which non-contiguous amino acids have been deleted relative to mature mammalian SEF protein are also envisioned.

Generally, mutants or variants of mammalian SEF proteins, encompassed by the present invention include natural or artificial variants differing by the addition, deletion and/or substitution of one or more contiguous or non-contiguous amino acid residues, or modified polypeptides in which one or more residues is modified, and mutants comprising one or more modified residues. In a one embodiment at least one, two, three, four, five, six, seven, eight, nine, or ten amino acids are deleted and/or substituted. Preferred mutants are natural or artificial variants of mammalian SEF proteins differing by the addition, deletion and/or substitution of one or more contiguous or non-contiguous amino acid residues. Such mutations can be in a conserved region or nonconserved region (compared to other SEF proteins), extracellular, cytoplasmic, or transmembrane region, for example.

In one embodiment, the protein includes an amino acid sequence having at least about 80%, at least about 85%, at least about 90%, at least about 95%, or at least about 98% amino acid sequence identity to SEQ ID NO:2. In another embodiment, the protein includes fragments or regions that have, at least about 90%, at least about 95%, 96% at least about, 97% at least about, 98% at least about, or at least about 99% amino acid sequence identity to the corresponding region of SEQ ID NO:2.

In a preferred embodiment, the SEF protein has an amino acid sequence shown in SEQ ID NO:2. In other embodiments, the SEF protein is substantially identical to SEQ ID NO:2. In yet another embodiment, the SEF protein is substantially identical to SEQ ID NO:2 and retains the functional activity of the protein of SEQ ID NO:2, as described in detail in the subsections above.

A “biologically active fragment or portion”, “biologically active mutant” and/or “biologically active fusion protein” of a mammalian SEF protein refers to an isolated and/or recombinant protein or oligopeptide which has at least one property, activity or function characteristic of a mammalian SEF (as defined herein) measured directly or indirectly such as a binding activity (e.g., FGF receptor binding activity), or a receptor tyrosine kinase regulatory function (e.g., inhibition of FGFR signaling). The SEF regulatory function can be measured directly or indirectly by measuring receptor signaling activity (e.g., inhibition of FGF signaling, inhibition of MAPK phosphorylation, and inhibition of FiRE activation) and, cellular response function (e.g., inhibition of proliferation, migration, angiogenesis). In a one embodiment biologically active fragments comprise about amino acid residues 353 to 739 of SEQ ID NO:2. In another embodiment, biologically active fragments comprise about amino acid residues 510 to 739 of SEQ ID NO:2.

Suitable fragments or mutants can be identified by screening. For example, the N-terminal, C-terminal, or internal regions of the protein can be deleted in a step-wise fashion and the resulting protein or polypeptide can be screened using a suitable assay, such as an assay described herein (e.g., FiRE luciferase assay). Where the resulting protein displays activity in the assay, the resulting protein (“fragment”) is functional.

In one embodiment the SEF protein, or fragment thereof, differs from the corresponding sequence in SEQ ID NO:2. In one embodiment it differs by at least one but by less than 15, 10 or 5 amino acid residues. In another embodiment, it differs from the corresponding sequence in SEQ ID NO:2 by at least one residue, but less than 20%, 15%, 10% or 5% of the residues in it differ from the corresponding sequence in SEQ ID NO:2. The differences are, preferably, differences or changes at a non-essential residue or a conservative substitution. In a preferred embodiment the differences are not in the intracellular domain (about residues 353 to 739 of SEQ ID NO:2) or the SH3 region (about amino acid residues 510 to 739). In another embodiment five or fewer conservative differences are in the intracellular domain (about residues 353 to 739 of SEQ ID NO:2) or the SH3 region (about amino acid residues 510 to 739).

In another aspect, the invention relates to an isolated SEF protein (e.g., mature SEF). In some embodiments, the isolated SEF protein has at least about 90% amino acid sequence identity with SEQ ID NO:2 and comprises one or more of the following: an arginine at a position corresponding to position 34 of SEQ ID NO:2, an alanine at a position corresponding to position 36 of SEQ ID NO:2, a glutamic acid at a position corresponding to position 248 of SEQ ID NO:2, a methionine at a position corresponding to position 255 of SEQ ID NO:2, a valine at a position corresponding to position 301 of SEQ ID NO:2, and an alanine at a position corresponding to position 608 of SEQ ID NO:2. The invention also relates to fragment of a SEF protein comprising an amino acid sequence selected from the group consisting of amino acid residues 36-739 of SEQ ID NO:2, amino acid residues 284-739 of SEQ ID NO:2, amino acid residues 36-353 of SEQ ID NO:2, and amino acid residues 36-510 of SEQ ID NO:2. The invention also relates to a fusion protein comprising a SEF protein or fragment as described herein and a second moiety operably linked thereto.

In another aspect the invention relates to fusion proteins. In one embodiment, the fusion protein comprises a SEF moiety, wherein the moiety has at least about 90% amino acid sequence identity with SEQ ID NO:2 and comprises one or more of the following: an arginine at a position corresponding to position 34 of SEQ ID NO:2, an alanine at a position corresponding to position 36 of SEQ ID NO:2, a glutamic acid at a position corresponding to position 248 of SEQ ID NO:2, a methionine at a position corresponding to position 255 of SEQ ID NO:2, a valine at a position corresponding to position 301 of SEQ ID NO:2, and an alanine at a position corresponding to position 608 of SEQ ID NO:2. The invention also relates to fragment of a SEF protein comprising an amino acid sequence selected from the group consisting of amino acid residues 36-739 of SEQ ID NO:2, amino acid residues 284-739 of SEQ ID NO:2, amino acid residues 36-353 of SEQ ID NO:2, and amino acid residues 36-510 of SEQ ID NO:2.

The term variant also encompasses fusion proteins, comprising mammalian SEF proteins (e.g., human SEF) as a first moiety, linked to a second moiety not occurring in the mammalian SEF as found in nature. Thus, the second moiety can be an amino acid, oligopeptide or polypeptide. The first moiety can be in an N-terminal location, C-terminal location or internal to the fusion protein. In one embodiment, the fusion protein comprises an affinity ligand (e.g., an enzyme, an antigen, epitope tag) as the first moiety, and a second moiety comprising a linker sequence and human SEF or portion thereof. Examples of SEF fusion proteins include SEF-FL FLAG, SEF-FL Myc (Examples 4-7).

It will be appreciated that isolated and/or recombinant mammalian SEF proteins and variants thereof can be modified, for example, by incorporation of or attachment (directly or indirectly (e.g., via a linker)) of a detectable label such as a radioisotope, spin label, antigen (e.g., epitope label such as a FLAG tag) or enzyme label, fluorescent or chemiluminesent group and the like, and such modified forms are included within the scope of the invention.

To determine the percent identity of two amino acid sequences, or of two nucleic acid sequences, the sequences are aligned for optimal comparison purposes (e.g., gaps can be introduced in one or both of a query and a reference amino acid or nucleic acid sequence for optimal alignment). In a preferred embodiment, the length of a query sequence aligned for comparison purposes is at least 30%, preferably at least 40%, more preferably at least 50%, even more preferably at least 60%, and even more preferably at least 70%, 80%, 90%, 100% of the length of the reference sequence (e.g., SEQ ID NO:1 or SEQ ID NO:2) The alignment identifies amino acids or nucleotides in the query sequence that correspond to those at defined positions in the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (1970) J. Mol. Biol. 48:444-453 algorithm which has been incorporated into the GAP program in the GCG software package (available at the bioinformatics page of the website maintained by Accelrys, Inc., San Diego, Calif., USA), using either a Blossum 62 matrix or a PAM250 matrix, and a gap weight of 16, 14, 12, 10, 8, 6, or 4 and a length weight of 1, 2, 3, 4, 5, or 6. A particularly preferred set of parameters are a Blossum 62 scoring matrix with a gap penalty of 12, a gap extend penalty of 4, and a frameshift gap penalty of 5.

In yet another preferred embodiment, the percent identity between two nucleotide sequences is determined using the GAP program in the GCG software package (available at the bioinformatics page of the website maintained by Accelrys, Inc., San Diego, Calif., USA), using a NWSgapdna.CMP matrix and a gap weight of 40, 50, 60, 70, or 80 and a length weight of 1, 2, 3, 4, 5, or 6. A particularly preferred set of parameters are a Blossum 62 scoring matrix with a gap penalty of 12, a gap extend penalty of 4, and a frameshift gap penalty of 5.

Other algorithms can also be used for example, the percent identity between two amino acid or nucleotide sequences can be determined using the algorithm of Meyers and Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a probe sequence to perform a search against public databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to SEF nucleic acid molecules of the invention. BLAST protein searches can be performed with the XBLAST program, score=50, wordlength=3 to obtain amino acid sequences homologous to SEF protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used (accessible at the website maintained by National Center for Biotechnology Information, Bethesda, Md., USA (ncbi.nlm.nih.gov)).

Nucleic Acids, Constructs and Vectors

The present invention relates to isolated and/or recombinant (including, e.g., essentially pure) nucleic acids having sequences which encode a mammalian (e.g., human) SEF protein or variant thereof as described herein. In a preferred embodiment the invention relates to isolated and/or recombinant (including, e.g., essentially pure) nucleic acids comprising the nucleotide sequence of SEQ ID NO:1 or the open reading frame thereof. Further preferred embodiments include isolated and/or recombinant nucleic acid encoding a SEF allele, wherein said SEF allele encodes a SEF protein having said amino acid sequence that contains an amino acid selected from the group consisting of an arginine that corresponds at position 34 of SEQ ID NO:2, an alanine that corresponds at position 36 of SEQ ID NO:2, a glutamic acid that corresponds at position 248 of SEQ ID NO:2, a methionine that corresponds at position 255 of SEQ ID NO:2, a valine that corresponds at position 301 of SEQ ID NO:2, and an alanine that corresponds at position 608 of SEQ ID NO:2.

In one embodiment, an isolated nucleic acid molecule of the invention includes the nucleotide sequence shown in SEQ ID NO:1, or a portion thereof, such as the coding region or open reading frame of SEQ ID NO:1. In a particular embodiment, the nucleic acid molecule comprises the coding region of SEQ ID NO:1 (e.g., nucleotides 90 to 2309 of SEQ ID NO:1). In a preferred embodiment, the nucleic acid molecule includes the coding sequence of SEQ ID NO:1 operatively liked to one or more control elements. In another embodiment, the nucleic acid molecule encodes a fragment of SEF comprising from about amino acid 36 to about amino acid 739 of SEQ ID NO:2, about amino acid 284 to about amino acid 739 of SEQ ID NO:2, about amino acid 36 to about amino acid 353 of SEQ ID NO:2 or about amino acid 36 to about amino acid 510 of SEQ ID NO:2.

In another embodiment, the invention relates to an isolated and/or recombinant nucleic acid that encodes a SEF protein or fragment as described herein. The isolated and/or recombinant nucleic acid can comprise a nucleotide sequence having at least about 90% nucleotide sequence identity with SEQ ID NO:1 or the coding sequence thereof, and contain one or more of the following: a cytosine at a position corresponding to position 100 of SEQ ID NO:1, a cytosine at a position corresponding to position 107 of SEQ ID NO:1, a guanine at a position corresponding to position 742 of SEQ ID NO:1, a thymine at a position corresponding to position 764 of SEQ ID NO:1, a guanine at a position corresponding to position 901 of SEQ ID NO:1, and a guanine at a position corresponding to position 1822 of SEQ ID NO:1.

Nucleic acids referred to herein as “isolated” are nucleic acids separated away from the nucleic acids of the genomic DNA or cellular RNA of their source of origin (e.g., as it exists in cells or in a mixture of nucleic acids such as a library), and may have undergone further processing. “Isolated” nucleic acids include nucleic acids obtained by methods described herein, similar methods or other suitable methods, including essentially pure nucleic acids, nucleic acids produced by chemical synthesis, by combinations of biological and chemical methods, and recombinant nucleic acids which are isolated. Nucleic acids referred to herein as “recombinant” are nucleic acids which have been produced by recombinant DNA methodology, including those nucleic acids that are generated by procedures which rely upon a method of artificial recombination, such as the polymerase chain reaction (PCR) and/or cloning into a vector using restriction enzymes. “Recombinant” nucleic acids are also those that result from recombination events that occur through the natural mechanisms of cells, but are selected for after the introduction to the cells of nucleic acids designed to allow and make probable a desired recombination event.

The invention further relates to isolated and/or recombinant nucleic acids, including double or single stranded DNA or RNA, that are characterized by (1) their ability to hybridize under high-stringency conditions to: (a) a nucleic acid having the sequence SEQ ID NO:1, (b) a nucleic acid having a sequence which is complementary to SEQ ID NO:1, or (c) a portion of the foregoing comprising the open reading frame of SEQ ID NO:1 (a portion of SEQ ID NO:1 or the corresponding portion of the complementary strand), but do not hybridize to SEQ ID NO:3. In other embodiments, the isolated nucleic acids are further characterized by their ability to encode a polypeptide having the amino acid sequence SEQ ID NO:2 or a biologically active equivalent thereof (e.g., binding activity (e.g., FGF receptor binding activity), or a receptor tyrosine kinase regulatory function (e.g., inhibition of FGF-R signaling). The SEF regulatory function can be measured directly or indirectly by measuring receptor signaling activity (e.g., inhibition of FGF signaling, inhibition of MAPK phosphorylation, and inhibition of FiRE activation) or inhibition of a resulting cellular response (e.g., inhibition of proliferation, migration, angiogenesis)).

In one embodiment, the percent amino acid sequence identity between SEQ ID NO:2 and a biological equivalent thereof is at least about 60% (≧60%). In a preferred embodiment, biological equivalents of SEQ ID NO:2 share at least about 70% sequence identity with SEQ ID NO:2. More preferably, the percent amino acid sequence identity between SEQ ID NO:2 and biological equivalents thereof is at least about 80%, more preferably, at least about 90%, and still more preferably at least about 95% or 99% amino acid sequence identity.

Isolated and/or recombinant nucleic acids meeting these criteria include nucleic acids having sequences identical to sequences of naturally occurring mammalian SEF and biologically active portions thereof, or variants of the naturally occurring sequences. Such variants include mutants differing by the addition, deletion or substitution of one or more residues, modified nucleic acids in which one or more residues is modified (e.g., DNA or RNA analogs), and mutants comprising one or more modified residues. In one embodiment an isolated nucleic acid molecule of the invention has a nucleotide sequence encoding a protein comprising a SEF, wherein the SEF has an amino acid sequence which differs by at least one but by less than 15, 10 or 5 amino acid residues. In another embodiment, the nucleic acid shares at least about 50% nucleotide sequence identity, more preferably at least about 75% nucleotide sequence identity, more preferably at least about 90% nucleotide sequence identity, and still more preferentially 95% nucleotide sequence identity and most preferentially 99.8% nucleotide sequence identity, with one strand of the sequence illustrated in SEQ ID NO:1 or to the coding region thereof. Preferred nucleic acids have lengths of at least about 40 nucleotides, more preferably at least about 50 nucleotides, and still more preferably at least about 75 nucleotides.

Such nucleic acids can be detected and isolated by hybridization under high stringency conditions. Exemplary high stringency conditions hybridize in 0.5M sodium phosphate, 7% SDS at 65° C., followed by one or more washes at 0.2×SSC, 1% SDS at 65° C. Factors such as probe length, base composition, percent mismatch between the hybridizing sequences, temperature and ionic strength influence the stability of nucleic acid hybrids. Thus, alternative high stringency hybridization conditions can be readily determined empirically.

Isolated and/or recombinant nucleic acids that are characterized by their ability to hybridize to a nucleic acid having the sequence SEQ ID NO:1 or the complement thereof (e.g., under high stringency conditions), may further encode a protein or polypeptide having at least one function characteristic of a mammalian SEF protein (e.g., a human SEF protein), such as binding activity (e.g., FGF receptor binding activity), or a receptor tyrosine kinase regulatory function (e.g., inhibition of FGF-R signaling). The SEF regulatory function can be measured directly or indirectly by measuring receptor signaling activity (e.g., inhibition of FGF signaling, inhibition of MAPK phosphorylation, and inhibition of FiRE activation) or inhibition of a resulting cellular response (e.g., inhibition of proliferation, migration, angiogenesis).

The SEF regulatory function can be measured directly or indirectly by measuring receptor signaling activity (e.g., inhibition of FGF signaling, inhibition of MAPK phosphorylation, and inhibition of FiRE activation) or cellular response function (e.g., inhibition of proliferation, migration, angiogenesis).

The human SEF nucleic acid described herein, or sufficient portions thereof, whether isolated, recombinant and/or synthetic, including fragments produced by PCR, can be used as probes or primers to detect and/or recover nucleic acids (e.g., genomic DNA, allelic variants, cDNA) encoding SEF (homologs) or other related SEF-like proteins (e.g., novel SEF genes) from other mammalian species including, but not limited to primates (e.g., a primate other than a human, such as a monkey (e.g., cynomolgus monkey)), bovine, ovine, equine, canine, feline, porcine, piscine, poultry (chicken), and rodent (e.g., guinea pig, murine species such as rat, mouse). This can be achieved using the procedures described herein or other suitable methods, including hybridization, PCR or other suitable techniques. Mammalian nucleic acids can be used to prepare constructs (e.g., vectors), receptor or fragments thereof, and host strains useful in the production and methods of use of receptor.

In one embodiment, a nucleic acid encoding a mammalian SEF protein (or variant) is producible by methods such as PCR amplification. For example, appropriate primers (e.g., a pair of primers or nested primers) can be designed which comprise a sequence which is complementary or substantially complementary to a portion of the human SEF cDNA described herein and others which comprise a sequence which has portion of the sequence of SEQ ID NO:1. For instance, primers complementary to and identical to the 5′- or 3′-ends of the coding sequence and/or flanking the coding sequence can be designed. Such primers can be used in a polymerase chain reaction with a suitable template nucleic acid to obtain nucleic acid encoding a mammalian SEF. Suitable templates include e.g., constructs described herein (such as SEF-FL), a cDNA or genomic library or another suitable source of mammalian (e.g., a human, primate) cDNA or genomic DNA. Primers can contain portions complementary to flanking sequences of a construct selected as template as appropriate.

In one embodiment, the nucleic acid or portion thereof encodes a protein or polypeptide having at least one function characteristic of a mammalian SEF protein (e.g., a human SEF protein), such as a binding activity (e.g., FGF receptor binding), or a receptor tyrosine kinase regulatory function (e.g., inhibition of FGFR signaling). The SEF regulatory function can be measured directly or indirectly by measuring receptor signaling activity (e.g., inhibition of FGF signaling, inhibition of MAPK phosphorylation, and inhibition of FiRE activation) and/or inhibition of a resulting cellular response (e.g., inhibition of proliferation, migration, angiogenesis). For example, as shown herein, a human SEF protein can inhibit FGF induced signaling in a mammalian cell (Examples 4 and 10). In one embodiment, proteins of the present invention can inhibit a FGF induced response, e.g., proliferation, migration, differentiation or embryonic patterning. The present invention also relates more specifically to isolated and/or recombinant nucleic acids or a portion thereof comprising sequences which encode a mammalian SEF or a portion thereof. The present invention relates even more specifically to isolated and/or recombinant nucleic acids comprising sequences which encode the human SEF protein comprising the amino acid sequence of SEQ ID NO:2.

The binding function of a protein or polypeptide (e.g., encoded by hybridizing nucleic acid) can be detected in binding or binding inhibition assays, using membrane fractions containing receptor or cells expressing receptor, for example (see e.g., Van Riper et al., J. Exp. Med., 177: 851-856 (1993); Sledziewski et al., U.S. Pat. No. 5,284,746 (Feb. 8, 1994)). In a preferred embodiment binding function of a protein includes forming a complex with an FGF receptor (e.g., FGFR1, FGFR2, FGFR3). Thus, the ability of the encoded protein or polypeptide to bind a ligand can be assessed. The antigenic properties of proteins or polypeptides encoded by nucleic acids of the present invention can be determined by immunological methods employing antibodies that bind to a mammalian SEF, such as immunoblotting, immunoprecipitation and immunoassay (e.g., radioimmunoassay, ELISA).

SEF regulatory function can be measured or detected directly or indirectly. For example, by measuring MAPK signaling (e.g., MAPK phosphorylation, FiRE activation as described in Examples 4 and 7), and/or cell proliferation in response to a suitable stimulus (e.g., FGF).

These methods, alone or in combination with other suitable methods can also be used in procedures for the identification and/or isolation of nucleic acids which encode a polypeptide having the amino acid sequence SEQ ID NO:2 or functional equivalents thereof, and having an activity detected by the assay. Portions of isolated nucleic acids which encode biologically active polypeptide portions of SEQ ID NO:2 having a certain function can be also identified and isolated in this manner.

Nucleic acids of the present invention can be used in the production of proteins or polypeptides. For example, a nucleic acid containing all or part of the coding sequence for a mammalian SEF (DNA which hybridizes to the sequence SEQ ID NO:1, or the complement thereof) can be incorporated into a construct for further manipulation of sequences or for production of the encoded polypeptide (e.g., in suitable host cells). Nucleic acids of the present invention can also be modified, for example, by incorporation of or attachment (directly or indirectly) of a detectable label such as a radioisotope, spin label, affinity label, epitope, enzyme, fluorescent group, chemiluminescent group and the like, and such modified forms are included within the scope of the invention.

Antisense Constructs

In another aspect, the invention is an antisense nucleic acid, which can hybridize with the target molecule. The target can be any nucleic acid encoding SEF. When introduced into a cell using suitable methods, antisense nucleic acid can inhibit the expression of the gene encoding SEF. Antisense nucleic acids can be produced using any suitable method.

In one embodiment, the antisense nucleic acid comprises at least twenty nucleotides and can hybridize with SEQ ID NO:1, wherein the target nucleic acid can hybridize to a nucleic acid having the sequence of the complement of SEQ ID NO:1. For example, antisense nucleic acid can be complementary to a target nucleic acid having the sequence of SEQ ID NO:1 or a portion thereof sufficient to allow hybridization. In another embodiment, the antisense nucleic acid is wholly or partially complementary to and can hybridize with a target nucleic acid which encodes a mammalian SEF (e.g., human SEF).

Antisense nucleic acids are useful for a variety of purposes, including research and therapeutic applications. For example, a construct comprising an antisense nucleic acid can be introduced into a suitable cell to inhibit SEF expression. Such a cell can be used, for instance, in assessing the biological functions of SEF. In another aspect, such a construct can be introduced into some or all of the cells of an animal (e.g., a mammal). The antisense nucleic acid inhibits SEF expression, and can ameliorate a SEF mediated disease, e.g., cardiovascular disease, kidney disease, cellular proliferative disease, and FGF receptor related diseases. Thus, a cardiovascular disease or condition can be treated using an antisense nucleic acid of the present invention. Suitable animals comprising an antisense construct can also provide useful models for conditions characterized by endothelial cell dysfunction and can provide further information regarding SEF function. Such animals can provide valuable models of kidney disorders, cellular proliferative disorders, FGF receptor related disorders, cardiovascular disorders, and are useful for elucidating the role of endothelial cells in normal and pathogenic processes.

Method of Producing Recombinant Proteins

Another aspect of the invention relates to a method of producing a mammalian SEF protein or variant (e.g., portion) thereof. Recombinant protein can be obtained, for example, by the expression of a recombinant nucleic acid (e.g., DNA) molecule encoding a mammalian SEF or variant thereof in a suitable host cell.

Constructs (e.g., expression vectors) suitable for the expression of a mammalian SEF protein or variant thereof are also provided. The constructs can be introduced into a suitable host cell, and cells which express a recombinant mammalian SEF protein or variant thereof can be produced and maintained in culture. Such cells are useful for a variety of purposes, including use in the production of protein for characterization, isolation and/or purification, (e.g., affinity purification), use as immunogen, and in FGF signaling assays or other functional assays (e.g., to screen for ligands, inhibitors and/or promoters of receptor function), for instance. Suitable host cells can be prokaryotic, including bacterial cells such as E. coli, B. subtilis and or other suitable bacteria, or eukaryotic, such as fungal or yeast cells (e.g., Pichia pastoris, Aspergillus species, Saccharomyces cerevisiae, Schizosaccharomyces pombe, Neurospora crassa), or Plant cells (e.g., tobacco) other lower eukaryotic cells, and cells of higher eukaryotes such as those from insects (e.g., Sf9 insect cells (WO 94/26087, O'Connor, published Nov. 24, 1994)) or mammals (e.g., endothelial cells, Chinese hamster ovary cells CHO(CCL-61, ATCC, Manassas, Va.), CV-1 origin, SV-40 (COS) cells, HuT 78 (TIB-161, ATCC, Manassas, Va.) cells, 293 cells (CRL-11268, ATCC, Manassas, Va.), and NIH3T3 (CRL-1658, ATCC, Manassas, Va.).

Host cells which produce a recombinant mammalian SEF protein or variant thereof can be produced using any suitable method. For example, a nucleic acid encoding all or part of the coding sequence for the desired protein can be inserted into a nucleic acid vector, e.g., a DNA vector, such as a plasmid, virus or other suitable replicon for expression. A variety of vectors are available, including vectors which are maintained in single copy or multiple copy, or which become integrated into the host cell chromosome.

Transcriptional and/or translational signals of a mammalian SEF gene can be used to direct expression. Suitable expression vectors for the expression of a nucleic acid encoding all or part of the coding sequence of the desired protein are also available. Suitable expression vectors can contain a number of components, including, but not limited to one or more of the following: an origin of replication; a selectable marker gene; one or more expression control elements, such as a transcriptional control element (e.g., a promoter, an enhancer, terminator), and/or one or more translation signals; a signal sequence or leader sequence for membrane targeting in a selected host cell (e.g., of mammalian origin or from a heterologous mammalian or non-mammalian species). In a construct, a signal sequence can be provided by the vector, the mammalian SEF coding sequence, or other source.

A promoter can be provided for expression in a suitable host cell. Promoters can be constitutive or inducible. In the vectors, a promoter can be operably linked to a nucleic acid encoding the mammalian SEF protein or variant thereof, such that it directs expression of the encoded polypeptide. A variety of suitable promoters for prokaryotic (e.g., lac, tac, T3, T7 promoters for E. coli) and eukaryotic (e.g., yeast alcohol dehydrogenase (ADH1), SV40, CMV) hosts are available.

In addition, the expression vectors typically comprise a selectable marker for selection of host cells carrying the vector, and, in the case of replicable expression vector, an origin or replication. Genes encoding products which confer antibiotic or drug resistance are common selectable markers and may be used in prokaryotic (e.g., β-lactamase gene (ampicillin resistance), Tet gene for tetracycline resistance) and eukaryotic cells (e.g., neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g., LEU2, URA3, HIS3) are often used as selectable markers in yeast. Use of viral (e.g., baculovirus) or phage vectors, and vectors which are capable of integrating into the genome of the host cell, such as retroviral vectors, are also contemplated. The present invention also relates to cells carrying these expression vectors.

For example, a nucleic acid encoding a mammalian SEF protein or variant thereof, or a construct comprising such nucleic acid, can be introduced into a suitable host cell by a method appropriate to the host cell selected (e.g., transformation, transduction, transfection, electroporation, infection), such that the nucleic acid is operably linked to one or more expression control elements (e.g., in a vector, in a construct created by processes in the cell, integrated into the host cell genome). Host cells can be maintained under conditions suitable for expression (e.g., in the presence of inducer, suitable media supplemented with appropriate salts, growth factors, antibiotic, nutritional supplements, etc.), whereby the encoded polypeptide is produced. If desired, the encoded protein (e.g., human SEF) can be isolated (e.g., from the host cells, medium, milk). It will be appreciated that the method encompasses expression in a host cell of a transgenic animal (see e.g., WO 92/03918, GenPharm International, published Mar. 19, 1992).

Fusion proteins also can be produced in this manner. For example, some embodiments can be produced by the insertion of a mammalian SEF protein cDNA or portion thereof into a suitable expression vector, such as BLUESCRIPT II SK +/−expression vector (Stratagene, La Jolla, Calif.), PGEX-4T-2 expression vector (Amersham Pharmacia Biotech, Piscataway, N.J.), PCDNA-3 expression vector (Invitrogen Life Technologies, Carlsbad, Calif.) or PET-15b expression vector (Novagen, Madison, Wis.). The resulting construct can be introduced into a suitable host cell for expression. Upon expression, fusion protein can be isolated or purified from a cell lysate by means of a suitable affinity matrix (see e.g., Current Protocols in Molecular Biology (Ausubel, F. M. et al., eds., Vol. 2, Suppl. 26, pp. 16.4.1-16.7.8 (1991)). In addition, affinity labels provide a means of detecting a fusion protein. For example, the cell surface expression or presence in a particular cell fraction of a fusion protein comprising an antigen or epitope affinity label can be detected by means of an appropriate antibody. Specific examples of SEF fusion proteins are described in the Materials and Method section of Example 4 to Example 7 contained herein.

Anti-SEF Antibodies

The antibody of the invention can be polyclonal or monoclonal, and the term “antibody” is intended to encompass both polyclonal and monoclonal antibodies. The terms polyclonal and monoclonal refer to the degree of homogeneity of an antibody preparation, and are not intended to be limited to particular methods of production. The term “antibody” as used herein encompasses antigen-binding fragments of antibodies, including antigen-binding fragments of human, humanized, chimeric, CDR-grafted, veneered or single-chain antibodies. In a preferred embodiment, the antibody has binding specificity for the polypeptide having the amino acid sequence of SEQ ID NO:2 and does not bind to the polypeptide having the amino acid sequence of SEQ ID NO:4.

Antibodies which bind SEF can be selected from a suitable collection of natural or artificial antibodies or raised against an appropriate immunogenic in a suitable host. For example, antibodies can be raised by immunizing a suitable host (e.g., mouse, human antibody-transgenic mouse) with a suitable immunogenic, such as an isolated or purified SEF or cells expressing a recombinant SEF (e.g., cell that expresses an exogenous nucleic acid encoding human SEF). In addition, cells expressing a recombinant SEF, such as transfected cells, can be used in a screen for antibody which binds thereto (See e.g., Chuntharapai et al., J. Immunol., 152: 1783-1789 (1994); Chuntharapai et al., U.S. Pat. No. 5,440,021).

Preparation of immunizing antigen, and polyclonal and monoclonal antibody production can be performed using any suitable technique. A variety of methods have been described. (See, e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al., Eds., (John Wiley & Sons: New York, N.Y.), Chapter 11, (1991).) Generally, where a monoclonal antibody is desired, a hybridoma is produced by fusing a suitable immortal cell line (e.g., a myeloma cell line such as SP2/0, P3X63Ag8.653 or a heteromyeloma) with antibody-producing cells. Antibody-producing cells can be obtained from the peripheral blood or, preferably the spleen or lymph nodes, of humans, human-antibody transgenic animals or other suitable animals immunized with the antigen of interest. Cells that produce antibodies of human origin (e.g., a human antibody) can be produced using suitable methods, for example, fusion of a human antibody-producing cell and a heteromyeloma or trioma, or immortalization of an activated human B cell via infection with Epstein Barr virus. (See, e.g., U.S. Pat. No. 6,197,582 (Trakht); Niedbala et al., Hybridoma, 17:299-304 (1998); Zanella et al., J Immunol Methods, 156:205-215 (1992); Gustafsson et al., Hum Antibodies Hybridomas, 2:26-32 (1991).) The fused or immortalized antibody-producing cells (hybridomas) can be isolated using selective culture conditions, and cloned by limiting dilution. Cells which produce antibodies with the desired specificity can be identified using a suitable assay (e.g., ELISA).

Other suitable methods of producing or isolating antibodies or antigen-binding fragments of the desired specificity can be used, including, for example, methods which select a recombinant antibody or antigen-binding fragment thereof from a library, such as a phage display library. Such libraries can contain antibodies or antigen-binding fragments of antibodies that contain natural or artificial amino acid sequences. For example, the library can contain Fab fragments which contain artificial CDRs (e.g., random amino acid sequences) and human framework regions. (See, for example, U.S. Pat. No. 6,300,064 (Knappik, et al.), the entire teachings of which are incorporated herein by reference.)

Human antibodies and nucleic acids encoding same can be obtained from a human or from human-antibody transgenic animals. Human-antibody transgenic animals (e.g., mice) are animals that are capable of producing a repertoire of human antibodies, such as XENOMOUSE (Abgenix, Fremont, Calif.), HUMAB-MOUSE, KIRIN TC MOUSE or KM-MOUSE (MEDAREX, Princeton, N.J.). Generally, the genome of human-antibody transgenic animals has been altered to include a transgene comprising DNA from a human immunoglobulin locus that can undergo functional rearrangement. An endogenous immunoglobulin locus in a human-antibody transgenic animal can be disrupted or deleted to eliminate the capacity of the animal to produce antibodies encoded by an endogenous gene. Suitable methods for producing human-antibody transgenic animals are well known in the art. (See, for example, U.S. Pat. Nos. 5,939,598 and 6,075,181 (Kucherlapati et al.), U.S. Pat. Nos. 5,569,825, 5,545,806, 5,625,126, 5,633,425, 5,661,016, and 5,789,650 (Lonberg et al.), Jakobovits et al., Proc. Natl. Acad. Sci. USA, 90: 2551-2555 (1993), Jakobovits et al., Nature, 362: 255-258 (1993), Jakobovits et al. WO 98/50433, Jakobovits et al. WO 98/24893, Lonberg et al. WO 98/24884, Lonberg et al. WO 97/13852, Lonberg et al. WO 94/25585, Lonberg et al. EP 0 814 259 A2, Lonberg et al. GB 2 272 440 A, Lonberg et al., Nature 368:856-859 (1994), Lonberg et al., Int Rev Immunol 13(1):65-93 (1995), Kucherlapati et al. WO 96/34096, Kucherlapati et al. EP 0 463 151 B 1, Kucherlapati et al. EP 0 710 719 A1, Surani et al. U.S. Pat. No. 5,545,807, Bruggemann et al. WO 90/04036, Bruggemann et al. EP 0 438 474 B1, Taylor et al., Int. Immunol. 6(4)579-591 (1994), Taylor et al., Nucleic Acids Research 20(23):6287-6295 (1992), Green et al, Nature Genetics 7:13-21 (1994), Mendez et al., Nature Genetics 15:146-156 (1997), Tuaillon et al., Proc Natl Acad Sci USA 90(8)3720-3724 (1993) and Fishwild et al., Nat Biotechnol 14(7):845-851 (1996), the teachings of each of the foregoing are incorporated herein by reference in their entirety.)

As described herein, human-antibody transgenic animals can be immunized with a suitable composition comprising an antigen of interest (e.g., a recombinant cell expressing SEF). Antibody producing cells can be isolated and fused to form hybridomas using conventional methods. Hybridomas that produce human antibodies having the desired characteristics (e.g., specificity, affinity) can be identified using any suitable assay (e.g, ELISA) and, if desired, selected and subcloned using suitable culture techniques.

Human-antibody transgenic animals provide a source of nucleic acids that can be enriched in nucleic acids that encode antibodies having desired properties, such as specificity and affinity. For example, nucleic acids encoding antibodies or antibody variable regions can be isolated from human-antibody transgenic mice that have been immunized with SEF. The isolated nucleic acids or portions thereof (e.g., portions encoding variable regions, CDRs, framework regions) can be expressed using any suitable method (e.g., phage display) to produce a library of antibodies or antigen-binding fragments of antibodies (e.g., single chain antigen-binding fragments, double chain antigen-binding fragments) that is enriched for antibodies or antigen-binding fragments that bind SEF. Such a library can exhibit enhanced diversity (e.g., combinatorial diversity through pairing of heavy chain variable regions and light chain variable regions) relative to the repertoire of antibodies produced in the immunized human-antibody transgenic animal. The library can be screened using any suitable assay (e.g., SEF binding assay) to identify antibodies or antigen-binding fragments having desired properties (e.g., specificity, affinity). The nucleic acids encoding antibody or antigen-binding fragments having desired properties can be recovered using any suitable methods. (See, e.g., U.S. Pat. No. 5,871,907 (Winter et al.) and U.S. Pat. No. 6,057,098 (Buechler et al.), the entire teachings of each of the foregoing are incorporated herein by reference.)

The antibody of the invention can be a CDR-grafted (e.g., humanized) antibody or an antigen-binding fragment thereof. The CDRs of a CDR-grafted antibody can be derived from a suitable antibody which binds SEF (referred to as a donor antibody). Other sources of suitable CDRs include natural and artificial SEF-specific antibodies obtained from nonhuman sources, such as rodent (e.g., mouse, rat), rabbit, pig, goat, non-human primate (e.g., monkey) or non-human library.

The framework regions of a CDR-grafted antibody are preferably of human origin, and can be derived from any human antibody variable region having sequence similarity to the analogous or equivalent region (e.g., light chain variable region) of the antigen binding region of the donor antibody. Other sources of framework regions of human origin include human variable region consensus sequences. (See, e.g., Kettleborough, C. A. et al., Protein Engineering 4:773-783 (1991); Carter et al., WO 94/04679; Kabat, E. A., et al., Sequences of Proteins of Immunological Interest, Fifth Edition, U.S. Department of Health and Human Services, U.S. Government Printing Office (1991)).

In one embodiment, the framework regions of a CDR-grafted (e.g., humanized) antibody chain can be derived from a variable region of human origin having at least about 65% overall amino acid sequence identity, and preferably at least about 70% overall amino acid sequence identity, with the amino acid sequence of the variable region of the donor antibody. A suitable framework region can also be derived from a antibody of human origin having at least about 65% amino acid sequence identity, and preferably at least about 70%, 80%, 90% or 95% amino acid sequence identity over the length of the framework region within the amino acid sequence of the equivalent portion (e.g., framework region) of the donor antibody. For example, a suitable framework region of human origin can be derived from an antibody of human origin (e.g., a human antibody) having at least about 65% amino acid sequence identity, and preferably at least about 70%, 80%, 90% or 95% amino acid sequence identity, over the length of the particular framework region being used, when compared to the amino acid sequence of the equivalent portion (e.g., framework region) of the donor antibody.

Framework regions of human origin can include amino acid substitutions or replacements, such as “back mutations” which replace an amino acid residue in the framework region of human origin with a residue from the corresponding position of the donor antibody. One or more mutations in the framework region can be made, including deletions, insertions and substitutions of one or more amino acids. Preferably, the CDR-grafted (e.g., humanized) antibody binds SEF with an affinity similar to, substantially the same as, or better than that of the donor antibody. Variants can be produced by a variety of suitable methods, including mutagenesis of nonhuman donor or acceptor human chains. (See, e.g., U.S. Pat. No. 5,693,762 (Queen et al.) and U.S. Pat. No. 5,859,205 (Adair et al.), the entire teachings of which are incorporated herein by reference.)

Constant regions of antibodies, antibody chains (e.g, heavy chain, light chain) or fragments or portions thereof of the invention, if present, can be derived from any suitable source. For example, constant regions of human, humanized and certain chimeric antibodies, antibody chains (e.g, heavy chain, light chain) or fragments or portions thereof, if present can be of human origin and can be derived from any suitable human antibody or antibody chain. For example, a constant region of human origin or portion thereof can be derived from a human κ or λ light chain, and/or a human γ (e.g., γ1, γ2, γ3, γ4), μ, α (e.g., α1, α2), δ or ε heavy chain, including allelic variants. In certain embodiments, the antibody or antigen-binding fragment (e.g., antibody of human origin, human antibody) can include amino acid substitutions or replacements that alter or tailor function (e.g., effector function). For example, a constant region of human origin (e.g., γ1 constant region, γ2 constant region) can be designed to reduce complement activation and/or Fc receptor binding. (See, for example, U.S. Pat. No. 5,648,260 (Winter et al.), U.S. Pat. No. 5,624,821 (Winter et al.) and U.S. Pat. No. 5,834,597 (Tso et al.), the entire teachings of which are incorporated herein by reference.) Preferably, the amino acid sequence of a constant region of human origin that contains such amino acid substitutions or replacements is at least about 95% identical over the full length to the amino acid sequence of the unaltered constant region of human origin, more preferably at least about 99% identical over the full length to the amino acid sequence of the unaltered constant region of human origin.

Humanized antibodies or antigen-binding fragments of a humanized antibody can be prepared using any suitable method. Several such methods are well-known in the art. (See, e.g., U.S. Pat. No. 5,225,539 (Winter), U.S. Pat. No. 5,530,101 (Queen et al.).) The portions of a humanized antibody (e.g., CDRs, framework, constant region) can be obtained or derived directly from suitable antibodies (e.g., by de novo synthesis of a portion), or nucleic acids encoding an antibody or chain thereof having the desired property (e.g., binds SEF) can be produced and expressed. Humanized immunoglobulins comprising the desired portions (e.g., CDR, FR, constant region) of human and nonhuman origin can be produced using synthetic and/or recombinant nucleic acids to prepare a nucleic acid (e.g., cDNA) encoding the desired humanized chain. To prepare a portion of a chain, one or more stop codons can be introduced at the desired position. For example, nucleic acid (e.g., DNA) sequences coding for newly designed humanized variable regions can be constructed using PCR mutagenesis methods to alter existing DNA sequences. (See, e.g., Kamman, M., et al., Nucl. Acids Res. 17:5404 (1989).) PCR primers coding for the new CDRs can be hybridized to a DNA template of a previously humanized variable region which is based on the same, or a very similar, human variable region (Sato, K., et al., Cancer Research 53:851-856 (1993)). If a similar DNA sequence is not available for use as a template, a nucleic acid comprising a sequence encoding a variable region sequence can be constructed from synthetic oligonucleotides (see e.g., Kolbinger, F., Protein Engineering 8:971-980 (1993)). A sequence encoding a signal peptide can also be incorporated into the nucleic acid (e.g., on synthesis, upon insertion into a vector). The natural signal peptide sequence from the acceptor antibody, a signal peptide sequence from another antibody or other suitable sequence can be used (see, e.g., Kettleborough, C. A., Protein Engineering 4:773-783 (1991)). Using these methods, methods described herein or other suitable methods, variants can be readily produced. In one embodiment, cloned variable regions can be mutated, and sequences encoding variants with the desired specificity can be selected (e.g., from a phage library; see, e.g., U.S. Pat. No. 5,514,548 (Krebber et al.) and WO 93/06213 (Hoogenboom et al.)).

The antibody of the invention can be a chimeric antibody or an antigen-binding fragment of a chimeric antibody. Preferably, the chimeric antibody or antigen-binding fragment thereof comprises a variable region of non-human origin and a constant region of human origin (e.g., a human constant region).

Chimeric antibodies and antigen-binding fragments of chimeric antibodies that bind SEF can be prepared using any suitable method. Several suitable methods are well-known in the art. (See, e.g., U.S. Pat. No. 4,816,567 (Cabilly et al.), U.S. Pat. No. 5,116,946 (Capon et al.).) Generally, chimeric antibodies are produced by preparing, for each of the light and heavy chain components of the chimeric immunoglobulin, a recombinant nucleic acid comprising a first nucleotide sequence encoding at least the variable region of an antibody from a first species that binds SEF that is joined in frame to a second nucleotide sequence encoding at least a part of a constant region from an antibody of a different species. Generally, the recombinant nucleic acid encodes a chimeric heavy chain or a chimeric light chain. However, if desired, a single recombinant nucleic acid encoding a chimeric heavy chain and a chimeric light chain can be prepared. The recombinant nucleic acids can be assembled in or inserted into an expression vector. The recombinant nucleic acid(s) can be introduced into a suitable host cell that is capable of expressing the chimeric antibody or chimeric antibody chain using any suitable method (e.g., transfection, transformation, infection) to produce a recombinant host cell. The recombinant host cell can be maintained under conditions suitable for expression of the chimeric antibody or chimeric antibody chain and the antibody or chain can be recovered.

Nucleic acids encoding the variable region of antibody light and heavy chains can be obtained from cells (e.g., B cells, hybridoma cells) that produce an antibody that binds SEF. Nucleic acids that encode constant regions can be obtained from suitable sources using any suitable technique, such a conventional techniques of recombinant DNA technology. The nucleotide sequences of nucleic acids encoding human κ or λ light chain constant regions, and γ (e.g., γ1, γ2, γ3, γ4), μ, α (e.g., α1, α2), δ or ε human heavy chain constant regions are readily available.

The invention also relates to a bispecific antibody or antigen-binding fragment thereof (e.g., F(ab′)₂), which binds SEF and at least one other antigen. Bispecific antibodies can be secreted by triomas and hybrid hybridomas. Generally, triomas are formed by fusion of a hybridoma and a lymphocyte (e.g., antibody secreting B cell) and hybrid hybridomas are formed by fusion of two hybridomas. Each of the cells that are fused to produce a trioma or hybrid hybridoma produces a monospecific antibody. However, triomas and hybrid hybridomas can produce an antibody containing antigen binding sites which recognize different antigens. The supernatants of triomas and hybrid hybridomas can be assayed for bispecific antibody using a suitable assay (e.g., ELISA), and bispecific antibodies can be purified using conventional methods. (See, e.g., U.S. Pat. No. 5,959,084 (Ring et al.) U.S. Pat. No. 5,141,736 (Iwasa et al.), U.S. Pat. Nos. 4,444,878, 5,292,668 and 5,523,210 (Paulus et al.) and U.S. Pat. No. 5,496,549 (Yamazaki et al.).)

The various portions of an antibody (e.g., mouse antibody, human antibody, humanized antibody, chimeric antibody and antigen-binding fragments of the foregoing) can be joined together chemically using conventional techniques, or can be prepared as a continuous polypeptide chain by expression (in vivo or in vitro) of a nucleic acid (one or more nucleic acids) encoding antibody. For example, nucleic acids encoding a human, humanized or chimeric chain can be expressed in vivo or in vitro to produce a continuous polypeptide chain. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 B1; and Padlan, E. A. et al., EP 0 519 596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single chain antibodies.

The invention also relates to antigen-binding fragments of antibodies that retain the capacity to bind antigen. Such antigen-binding fragments of antibodies retain the antigen binding function of a corresponding full-length antibody, and preferably inhibit binding of ligand to SEF. Antigen-binding fragments of antibodies encompassed by the invention include, Fv fragments (e.g., single chain Fv fragments (scFv)), Fab fragments, Fab′ fragments and F(ab′)₂ fragments, for example. Such antigen-binding fragments can be produced using any suitable method, for example by enzymatic cleavage and/or using recombinant DNA technology. For example, an antibody can be cleaved with papain or pepsin to yield a Fab fragment or F(ab′)₂ fragment, respectively. Other proteases with the requisite substrate specificity can also be used to generate antigen-binding fragments of antibodies, such as Fab fragments or F(ab′)₂ fragments. Similarly, Fv fragments can be prepared by digesting an antibody with a suitable protease or using recombinant DNA technology. For example, a nucleic acid can be prepared that encodes a light chain variable region and heavy chain variable region that are connected by a suitable peptide linker, such as a chain of two to about twenty Glycyl residues. The nucleic acid can be introduced into a suitable host (e.g., E. coli) using any suitable technique (e.g., transfection, transformation, infection), and the host can be maintained under conditions suitable for expression of a single chain Fv fragment. A variety of antigen-binding fragments of antibodies can be prepared using antibody genes in which one or more stop codons has been introduced upstream of the natural stop site. For example, an expression construct encoding a F(ab′)₂ portion of an immunoglobulin heavy chain can be designed by introducing a translation stop codon at the 3′ end of the sequence encoding the hinge region of the heavy chain.

The invention also relates to the individual heavy and light chains of the antibodies (e.g., mouse antibodies, human antibodies, humanized antibodies, chimeric antibodies) that bind SEF and to antigen-binding portions thereof. The heavy chains or light chains (and antigen-binding portions thereof) of the invention can bind SEF when paired with a complementary light or heavy chain, respectively. Complementary chains can be identified using any suitable method (e.g., phage display, transgenic animals). For example, a transgenic animal comprising a functionally rearranged nucleic acid encoding a desired heavy chain can be prepared. The heavy-chain transgenic animal can be immunized with the antigen of interest and hybridomas produced. Because of allelic exclusion at immunoglubulin loci, the heavy-chain transgenic mouse may not significantly express endogenous heavy chains and substantially all antibodies elicited by immunization can comprise the heavy chain of interest and a complementary light chain.

Fragments of SEF which include amino acids 36 to 56, 66 to 86, 96 to 106, 110 to 130, 150 to 170, 190 to 210, 230 to 250 or 260 to 280 of SEQ ID NO:2 can be used to make an antibody that binds an extracellular region of the SEF protein; fragments of SEF which include residues 310 to 330, 350 to 370, 390 to 410, 450 to 470, 490 to 510, 550 to 570, 600 to 620, 630 to 650, 700 to 720, or 721 to 739 of SEQ ID NO:2 can be used to make an antibody that binds an intracellular region of the SEF protein. In a preferred embodiment, antibodies that have binding specificity or that selectively bind any of these regions or other regions or domains of SEF and that do not bind SEQ ID NO:4, described herein are provided.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994) Bio/Technology 12:899-903).

The anti-SEF antibody can be a single chain antibody. A single-chain antibody (scFV) can be engineered as described in, for example, Colcher et al. (1999) Ann. N Y Acad. Sci. 880:263-80; and Reiter (1996) Clin. Cancer Res. 2:245-52. The single chain antibody can be dimerized or multimerized to generate multivalent antibodies having specificities for different epitopes of the same target SEF protein.

An antibody (or fragment thereof) may be conjugated to a therapeutic moiety such as a cytotoxin, a therapeutic agent or a radioactive ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids). Radioactive ions include, but are not limited to iodine, yttrium and praseodymium.

The conjugates of the invention can be used for modifying a given biological response, the therapeutic moiety is not to be construed as limited to classical chemical therapeutic agents. For example, the therapeutic moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Identification of Ligands, Inhibitors or Promoters of SEF Function

As used herein, a ligand is a substance which binds to a SEF protein (e.g., an FGF receptor, FGFR1, FGFR2, FGFR3). A ligand of a selected mammalian SEF protein is a substance which binds to the selected mammalian SEF protein. In a preferred embodiment, ligand binding of a mammalian SEF protein occurs with high affinity. As used herein “high affinity” means that at equilibrium ligand receptor binding is favored. In preferred embodiments the ligand binds with an affinity of at least 10⁻³M, 10⁻⁴M, 10⁻⁵M, 10⁻⁶M, 10⁻⁷M, 10⁻⁸M, 10⁻⁹M. In one embodiment the high affinity ligand is the FGFR. The term ligand refers to substances including, but not limited to, a natural ligand, whether isolated and/or purified, synthetic, and/or recombinant, a homolog of a natural ligand (e.g., from another mammal), antibodies, portions of such molecules, and other substances which bind SEF. The term ligand encompasses substances which are inhibitors or promoters of SEF activity, as well as substances which bind SEF, but lack inhibitor or promoter activity.

As used herein, an inhibitor is a substance which inhibits at least one function characteristic of a mammalian SEF protein (e.g., a human SEF), such as a binding activity (e.g., FGFR binding), or a receptor tyrosine kinase regulatory function (e.g., inhibition of FGFR signaling). The SEF regulatory function can be measured directly or indirectly, for example by measuring receptor signaling activity (e.g., inhibition of FGF signaling, inhibition of MAPK phosphorylation, and inhibition of FiRE activation) and/or, cellular response (e.g., inhibition of proliferation, migration, angiogenesis). The term inhibitor refers to a substance including an antagonist which binds SEF (e.g., an antibody, a mutant of a natural ligand, other competitive inhibitor of ligand binding), and a substance which inhibits SEF function without binding thereto.

As used herein, a promoter is a substance which promotes (induces or enhances) at least one function characteristic of a mammalian SEF protein (e.g., a human SEF), such as a binding activity (e.g., ligand, inhibitor and/or promoter binding), regulatory activity (e.g., inhibition of FGF signaling, inhibition of MAPK phosphorylation, and inhibition of FiRE activation) and, cellular response function (e.g., inhibition of proliferation, migration, angiogenesis). The term promoter refers to a substances including an agonist which binds SEF (e.g., an antibody, a homolog of a natural ligand from another species), and a substance which promotes SEF function without binding thereto (e.g., by activating an associated protein).

The assays described below, which employ the nucleic acids and proteins of the present invention, can be used, alone or in combination with each other or other suitable methods, to identify ligands, inhibitors or promoters of a mammalian SEF protein or variant. The in vitro methods of the present invention can be adapted for high-throughput screening in which large numbers of samples are processed (e.g., a 96 well format). Host cells comprising a nucleic acid of the present invention and expressing recombinant mammalian SEF (e.g., human SEF) at levels suitable for high-throughput screening can be used, and thus, are particularly valuable in the identification and/or isolation of ligands, inhibitors and promoters of mammalian SEF proteins. Expression of SEF can be monitored in a variety of ways. For instance, expression can be monitored using antibodies of the present invention which bind SEF or a portion thereof. Also, commercially available antibodies can be used to detect expression of an antigen- or epitope-tagged fusion protein comprising a SEF protein or polypeptide (e.g., FLAG tagged SEF), and cells expressing the desired level can be selected.

Nucleic acid encoding a mammalian SEF protein, can be incorporated into an expression system to produce a SEF protein or polypeptide as described above. An isolated and/or recombinant SEF protein or polypeptide, such as a SEF expressed in cells stably or transiently transfected with a construct comprising a nucleic acid of the present invention, or in a cell fraction containing SEF (e.g., a membrane fraction from transfected cells, liposomes incorporating SEF), can be used in tests for SEF function. The SEF can be further purified if desired. Testing of SEF function can be carried out in vitro or in vivo.

An isolated, recombinant mammalian SEF protein, such as a human SEF as shown in SEQ ID NO:2, can be used in the present method, in which the effect of a compound is assessed by monitoring SEF function as described herein or using other suitable techniques. For example, stable or transient transfectants such as those described in Examples 2 to 4, or other suitable cells (e.g., baculovirus infected Sf9 cells, stable tranfectants of NIH 3T3 cells) can be used in functional assays.

According to the method of the present invention, compounds can be individually screened or one or more compounds can be tested simultaneously according to the methods herein. Where a mixture of compounds is tested, the compounds selected by the processes described can be separated (as appropriate) and identified by suitable methods (e.g., PCR, sequencing, chromatography). The presence of one or more compounds (e.g., a ligand, inhibitor, promoter) in a test sample can also be determined according to these methods.

Large combinatorial libraries of compounds (e.g., organic compounds, recombinant or synthetic peptides, “peptoids”, nucleic acids) produced by combinatorial chemical synthesis or other methods can be tested (see e.g., Zuckerman, R. N. et al., J. Med. Chem., 37: 2678-2685 (1994) and references cited therein; see also, Ohlmeyer, M. H. J. et al., Proc. Natl. Acad. Sci. USA 90:10922-10926 (1993) and DeWitt, S. H. et al., Proc. Natl. Acad. Sci. USA 90:6909-6913 (1993), relating to tagged compounds; Rutter, W. J. et al. U.S. Pat. No. 5,010,175; Huebner, V. D. et al., U.S. Pat. No. 5,182,366; and Geysen, H. M., U.S. Pat. No. 4,833,092). Where compounds selected from a combinatorial library by the present method carry unique tags, identification of individual compounds by chromatographic methods is possible.

In one embodiment, phage display methodology is used. For example, SEF can be contacted with a phage (e.g., a phage or collection of phage such as a library) displaying a polypeptide under conditions appropriate for SEF binding (e.g., in a suitable binding buffer). Phage bound to SEF can be selected using standard techniques or other suitable methods. Phage can be separated from SEF using a suitable elution buffer. For example, a change in the ionic strength or pH can lead to a release of phage. Alternatively, the elution buffer can comprise a release component or components designed to disrupt binding of compounds (e.g., one or more compounds which can disrupt binding of the displayed peptide to SEF, such as a ligand, inhibitor, and/or promoter which competitively inhibits binding). Optionally, the selection process can be repeated or another selection step can be used to further enrich for phage which bind SEF. The displayed polypeptide can be characterized (e.g., by sequencing phage DNA). The polypeptides identified can be produced and further tested for ligand binding, inhibitor and/or promoter function. Analogs of such peptides can be produced which will have increased stability or other desirable properties.

In one embodiment, phage expressing and displaying fusion proteins comprising a coat protein with an N-terminal peptide encoded by random sequence nucleic acids can be produced. Suitable host cells expressing a SEF protein or polypeptide of the present invention are contacted with the phage, bound phage are selected, recovered and characterized. (See e.g., Doorbar, J. and G. Winter, J. Mol. Biol., 244: 361 (1994) discussing a phage display procedure used with a G protein-coupled receptor).

Other sources of potential ligands, inhibitors and/or promoters of mammalian SEF proteins include, but are not limited to, variants of SEF ligands, including naturally occurring, synthetic or recombinant variants, other inhibitors and/or promoters (e.g., anti-SEF antibodies, antagonists, agonists), inhibitors and/or promoters (e.g., antagonists or agonists), and soluble portions of a mammalian SEF receptor, such as a suitable SEF peptide or analog which can inhibit SEF function (see e.g., Murphy, R. B., WO 94/05695).

Signaling Assays

The induction of a SEF regulatory function by an agent can be measured directly or indirectly by measuring receptor signaling activity (e.g., inhibition of FGF signaling, inhibition of MAPK phosphorylation, and inhibition of FiRE activation) and, cellular response function (e.g., inhibition of proliferation, migration, signaling function) by an agent can be monitored using any suitable method.

Screening Assays:

The invention provides methods (also referred to herein as “screening assays”) for identifying modulators, i.e., candidate or test compounds or agents (e.g., proteins, peptides, peptidomimetics, peptoids, small molecules or other drugs) which bind to SEF proteins, have a stimulatory or inhibitory effect on, for example, SEF expression or SEF activity, or have a stimulatory or inhibitory effect on, for example, the expression or activity of a SEF substrate. Compounds thus identified can be used to modulate the activity of target gene products (e.g., SEF genes) in a therapeutic protocol, to elaborate the biological function of the target gene product, or to identify compounds that disrupt normal target gene interactions.

The invention provides methods (also referred to herein as “screening assays”) for identifying modulators, i.e., candidate or test compounds or agents (e.g., proteins, peptides, peptidomimetics, peptoids, small molecules or other drugs) which bind to SEF proteins, have a stimulatory or inhibitory effect on, for example, SEF expression or SEF activity, or have a stimulatory or inhibitory effect on, for example, the expression or activity of a SEF substrate. Compounds thus identified can be used to modulate the activity of target gene products (e.g., SEF genes) in a therapeutic protocol, to elaborate the biological function of the target gene product, or to identify compounds that disrupt normal target gene interactions.

In one embodiment, the invention provides assays for screening candidate or test compounds which are substrates of a SEF protein or polypeptide or a biologically active portion thereof. In another embodiment, the invention provides assays for screening candidate or test compounds which bind to or modulate the activity of a SEF protein or polypeptide or a biologically active portion thereof.

In one embodiment, the invention provides a method for identifying a compound which modulates the ability of SEF to regulate MAPK phosphorylation. In another embodiment, the invention provides a method for identifying a compound which modulates the ability of SEF to regulate FGFR signaling or FGF-induced cellular proliferation. In the foregoing methods, the method can comprise the steps of combining a composition comprising a polypeptide having SEF or a biologically active fragment or variant thereof, e.g. the amino acid sequence of SEQ ID NO:2, with a test compound and detecting a change in SEF activity. For example, the assay can identify a compound which modulates, e.g. increases or decreases, MAPK phosphorylation, e.g. activates or inactivates an FGF-induced response element, e.g. linked to a detectable reporter, e.g. luciferase, or e.g. increases or decreases FGF-induced cellular response, e.g. cellular proliferation.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909-13; Erb et al. (1994) Proc. Natl. Acad. Sci. USA 91:11422426; Zuckermann et al. (1994). J. Med. Chem. 37:2678-85; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233-51.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one embodiment, an assay is a cell-based assay in which a cell which expresses a SEF protein or biologically active portion thereof is contacted with a test compound, and the ability of the test compound to modulate SEF regulatory activity is determined. Determining the ability of the test compound to modulate SEF regulatory activity can be accomplished by detecting or measuring, directly or indirectly for example, MAPK phosphorylation, cellular proliferation, FiRE activation. The cell, for example, can be of mammalian origin, e.g., human.

The ability of the test compound to modulate SEF binding to a compound, e.g., a SEF substrate or to a ligand, e.g. FGFR, or to bind to SEF can also be evaluated. This can be accomplished, for example, by coupling the compound, e.g., the substrate, with a radioisotope or enzymatic label such that binding of the compound, e.g., the substrate, to SEF can be determined by detecting the labeled compound, e.g., substrate, in a complex. Alternatively, SEF could be coupled with a radioisotope or enzymatic label to monitor the ability of a test compound to modulate SEF binding to a SEF substrate in a complex. For example, compounds (e.g., SEF substrates) can be labeled with ¹²⁵I, ¹⁴C, ³⁵S or ³H, either directly or indirectly, and the radioisotope detected by direct counting of radioemmission or by scintillation counting. If desired, compounds can be enzymatically labeled with, for example, horseradish peroxidase, alkaline phosphatase, or luciferase, and the enzymatic label detected by determination of conversion of an appropriate substrate to product.

The ability of a compound (e.g., a SEF substrate) to interact with SEF with or without the labeling of any of the interactants can be evaluated. For example, a microphysiometer can be used to detect the interaction of a compound with SEF without the labeling of either the compound or the SEF. McConnell et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and SEF.

In yet another embodiment, a cell-free assay is provided in which a SEF protein or biologically active portion thereof is contacted with a test compound and the ability of the test compound to bind to the SEF protein or biologically active portion thereof is evaluated. Preferred biologically active portions of the SEF proteins to be used in assays of the present invention include fragments which participate in interactions with non-SEF molecules, e.g., fragments with high surface probability scores.

Soluble and/or membrane-bound forms of isolated proteins (e.g., SEF proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, TRITON X-100 (t-Octylphenoxypolyethoxy ethanol, Sigma-Aldrich), TRITON X-114 (Polyethylene glycol tert-octylphenyl ether, Sigma-Aldrich) THESIT (Polyethylene glycol 400 dodecyl ether, Sigma-Aldrich), Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule can simply utilize the natural fluorescent energy of tryptophan residues. Labels are chosen that emit different wavelengths of light, such that the ‘acceptor’ molecule label can be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the SEF protein to bind to a target molecule can be accomplished using “Surface plasmon resonance” (see, e.g., Sjolander and Urbaniczky (1991) Anal. Chem. 63:2338-2345 and Szabo et al. (1995) Curr. Opin. Struct. Biol. 5:699-705).

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either SEF, an anti-SEF antibody or its target molecule to facilitate separation of complexed from uncomplexed forms of one or both of the proteins, as well as to accommodate automation of the assay. Binding of a test compound to a SEF protein, or interaction of a SEF protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/SEF fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or SEF protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of SEF binding or activity determined using standard techniques.

Other techniques for immobilizing either a SEF protein or a target molecule on matrices include using conjugation of biotin and streptavidin. Biotinylated SEF protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific or selective for the immobilized component (the antibody, in turn, can be directly labeled or indirectly labeled with, e.g., a labeled anti-Ig antibody).

In one embodiment, this assay is performed utilizing antibodies reactive with SEF protein or target molecules but which do not interfere with binding of the SEF protein to its target molecule. Such antibodies can be derivatized to the wells of the plate, and unbound target or SEF protein trapped in the wells by antibody conjugation. Methods for detecting such complexes, in addition to those described above for the GST-immobilized complexes, include immunodetection of complexes using antibodies reactive with the SEF protein or target molecule, as well as enzyme-linked assays which rely on detecting an enzymatic activity associated with the SEF protein or target molecule.

Alternatively, cell free assays can be conducted in a liquid phase. In such an assay, the reaction products are separated from unreacted components, by any of a number of suitable techniques, including but not limited to: differential centrifugation (see, for example, Rivas and Minton (1993) Trends Biochem Sci 18:284-7); chromatography (gel filtration chromatography, ion-exchange chromatography); electrophoresis (see, e.g., Ausubel et al., eds. (1999) Current Protocols in Molecular Biology, J. Wiley, New York.); and immunoprecipitation (see, for example, Ausubel et al., eds. (1999) Current Protocols in Molecular Biology, J. Wiley, New York). Suitable resins and chromatographic techniques are known to one skilled in the art (see, e.g., Heegaard (1998) J Mol Recognit 11:141-8; Hage and Tweed (1997) J Chromatogr B Biomed Sci Appl. 699:499-525). Further, fluorescence energy transfer can also be conveniently utilized, as described herein, to detect binding without further purification of the complex from solution.

In a preferred embodiment, the assay includes contacting the SEF protein or biologically active portion thereof with a compound which binds SEF to form an assay mixture, contacting the assay mixture with a test compound, and determining the ability of the test compound to interact with a SEF protein, wherein determining the ability of the test compound to interact with a SEF protein includes determining the ability of the test compound to preferentially bind to SEF or biologically active portion thereof, or to modulate the activity of a target molecule, as compared to the known compound.

As used herein “target gene products” are SEF polypeptides, proteins, and nucleic acids. The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The preferred target genes/products for use in this embodiment are the SEF proteins herein identified. In an alternative embodiment, the invention provides methods for determining the ability of the test compound to modulate the activity of a SEF protein through modulation of the activity of a downstream effector. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific or selective for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific or selective for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

In another embodiment, the reaction can be conducted in a liquid phase in the presence or absence of the test compound, the reaction products separated from unreacted components, and complexes detected; e.g., using an immobilized antibody specific or selective for one of the binding components to anchor any complexes formed in solution, and a labeled antibody specific or selective for the other partner to detect anchored complexes. Again, depending upon the order of addition of reactants to the liquid phase, test compounds that inhibit complex or that disrupt preformed complexes can be identified.

In another embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the SEF proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with SEF (“SEF-binding proteins” or “SEF-bp”) and are involved in SEF activity. Such SEF-bps can be activators or inhibitors of signals by the SEF proteins or SEF targets as, for example, downstream elements of a SEF-mediated signaling pathway.

The two-hybrid system is based on the modular nature of most transcription factors, which consist of separable DNA-binding and activation domains. Briefly, the assay utilizes two different DNA constructs. In one construct, the gene that codes for a SEF protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: SEF protein can be the fused to the activator domain.) If the “bait” and the “prey” proteins are able to interact, in vivo, forming a SEF-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the SEF protein.

In another embodiment, modulators of SEF expression are identified. For example, a cell or cell free mixture is contacted with a candidate compound and the expression of SEF mRNA or protein evaluated relative to the level of expression of SEF mRNA or protein in the absence of the candidate compound. When expression of SEF mRNA or protein is greater in the presence of the candidate compound than in its absence, the candidate compound is identified as a stimulator of SEF mRNA or protein expression. Alternatively, when expression of SEF mRNA or protein is less (statistically significantly less) in the presence of the candidate compound than in its absence, the candidate compound is identified as an inhibitor of SEF mRNA or protein expression. The level of SEF mRNA or protein expression can be determined by methods described herein for detecting SEF mRNA or protein.

In another aspect, the invention pertains to a combination of two or more of the assays described herein. For example, a modulating agent can be identified using a cell-based or a cell free assay, and the ability of the agent to modulate the activity of a SEF protein can be confirmed in vivo, e.g., in an animal such as an animal model for aberrant cellular proliferation or cardiovascular disorders.

This invention further pertains to novel agents identified by the above-described screening assays. Accordingly, it is within the scope of this invention to further use an agent identified as described herein (e.g., a SEF modulating agent, an antisense SEF nucleic acid molecule, a SEF-specific antibody, or a SEF-binding partner) in an appropriate animal model to determine the efficacy, toxicity, side effects, or mechanism of action, of treatment with such an agent. Furthermore, novel agents identified by the above-described screening assays can be used for treatments as described herein.

Diagnostic Application

The present invention relates to diagnostic methods. For example, a mutation(s) in a gene encoding a mammalian SEF protein can cause a defect in at least one function of SEF, thereby reducing or enhancing receptor function. For instance, a mutation which produces a variant of SEF or alters the level of expression, can reduce or enhance SEF function, reducing or enhancing processes mediated by SEF (e.g., regulation of FGF signaling). The presence of such a mutation can be determined using methods which detect or measure the presence of SEF function in cells (e.g., endothelial cells) of an individual or in a receptor preparation isolated from such cells. In these assays, reduced or enhanced levels of SEF and/or reduced or enhanced SEF function can be assessed.

The results herein (Example 10) indicate SEF is differentially expressed in cancer (e.g., breast cancer, ovarian cancer, prostate cancer) and therefore can be used to diagnose cancer (e.g., breast cancer, ovarian cancer, prostate cancer). In a preferred embodiment the invention is a diagnostic for breast cancer. In other embodiment the present invention is a diagnostic for ovarian cancer, colon cancer or prostate cancer.

Predictive Medicine

The present invention also pertains to the field of predictive medicine in which diagnostic assays, prognostic assays, and monitoring clinical trials are used for prognostic (predictive) purposes to thereby treat an individual.

Generally, the invention provides, a method of determining if a subject is at risk for a disorder related to a lesion in or the misexpression of a gene which encodes SEF.

Such disorders include, e.g., a disorder associated with the misexpression of SEF gene; a kidney disorder, a cellular proliferative disorder, a FGF receptor related disorder, and a cardiovascular disorder. In a preferred embodiment the disorder is breast cancer.

The method includes one or more of the following:

detecting, in a tissue of the subject, the presence or absence of a mutation which affects the expression of the SEF gene, or detecting the presence or absence of a mutation in a region which controls the expression of the gene, e.g., a mutation in the 5′ control region;

detecting, in a tissue of the subject, the presence or absence of a mutation which alters the structure of the SEF gene;

detecting, in a tissue of the subject, the misexpression of the SEF gene, at the mRNA level;

detecting, in a tissue of the subject, the misexpression of the gene, at the protein level.

In one embodiments the method includes: ascertaining the existence of at least one of: a deletion of one or more nucleotides from the SEF gene; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from SEQ ID NO:1 under highly stringent conditions, or naturally occurring mutants thereof or 5′ or 3′ flanking sequences naturally associated with the SEF gene; (ii) exposing the probe/primer to nucleic acid of the tissue; and detecting, by hybridization, e.g., in situ hybridization, of the probe/primer to the nucleic acid, the presence or absence of the genetic lesion.

In preferred embodiments detecting the misexpression includes ascertaining the existence of an alteration in the level of a messenger RNA transcript of the SEF gene.

Methods of the invention can be used prenatally or to determine if a subject's offspring will be at risk for a disorder.

In one embodiment, the method includes determining the structure of a SEF gene, an abnormal structure being indicative of risk for the disorder.

In preferred embodiments the method includes contacting a sample from the subject with an antibody to the SEF protein or a nucleic acid, which hybridizes specifically with the gene. These and other embodiments are discussed below.

Diagnostic and Prognostic Assays

The presence, level, or absence of SEF protein or nucleic acid in a biological sample can be evaluated by obtaining a biological sample from a test subject and contacting the biological sample with a compound or an agent capable of detecting SEF protein or nucleic acid (e.g., mRNA, genomic DNA) that encodes SEF protein such that the presence of SEF protein or nucleic acid is detected in the biological sample. The term “biological sample” includes tissues, cells and biological fluids isolated from a subject, as well as tissues, cells and fluids present within a subject. A preferred biological sample tissue (e.g., breast tissue). The level of expression of the SEF gene can be measured in a number of ways, including, but not limited to: measuring the mRNA encoded by the SEF genes; measuring the amount of protein encoded by the SEF genes; or measuring the activity of the protein encoded by the SEF genes.

The level of mRNA corresponding to the SEF gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length SEF nucleic acid, such as the nucleic acid of SEQ ID NO:1, or a portion thereof, such as an oligonucleotide of at least 7, 15, 30, 50, 100, 250 or 500 nucleotides in length and sufficient to specifically hybridize under stringent conditions to SEF mRNA or genomic DNA. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In another, the probes are immobilized on a surface and the mRNA (or cDNA) is contacted with the probes, for example, in a two-dimensional gene chip array. A skilled artisan can adapt any suitable mRNA detection methods for use in detecting the level of mRNA encoded by the SEF genes.

The level of mRNA in a sample that encodes a SEF protein can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al., (1989), Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the SEF gene being analyzed.

In another embodiment, the methods further contacting a control sample with a compound or agent capable of detecting SEF mRNA, or genomic DNA, and comparing the presence of SEF mRNA or genomic DNA in the control sample with the presence of SEF mRNA or genomic DNA in the test sample.

A variety of methods can be used to determine the level of protein encoded by SEF. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect SEF protein in a biological sample in vitro as well as in vivo. In vitro techniques for detection of SEF protein include enzyme linked immunosorbent assays (ELISAs), immunoprecipitations, immunofluorescence, enzyme immunoassay (EIA), radioimmunoassay (RIA), and Western blot analysis. In vivo techniques for detection of SEF protein include introducing into a subject a labeled anti-SEF antibody. For example, the antibody can be labeled with a radioactive marker whose presence and location in a subject can be detected by standard imaging techniques.

In another embodiment, the methods further include contacting the control sample with a compound or agent capable of detecting SEF protein, and comparing the presence of SEF protein in the control sample with the presence of SEF protein in the test sample.

The invention also includes kits for detecting the presence of SEF in a biological sample. For example, the kit can include a compound or agent capable of detecting SEF protein or mRNA in a biological sample; and a standard. The compound or agent can be packaged in a suitable container. The kit can further comprise instructions for using the kit to detect SEF protein or nucleic acid.

As used herein a “marker of the invention” is a SEF nucleic acid or SEF polypeptide that can be used to diagnose a disease (e.g., breast cancer). For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein stabilizing agent. The kit can also includes components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with aberrant or unwanted SEF expression or activity. As used herein, the term “unwanted” includes an unwanted phenomenon involved in a biological response such as pain or deregulated cell proliferation.

In one embodiment, a disease or disorder associated with aberrant or unwanted SEF expression or activity is identified. A test sample is obtained from a subject and SEF protein or nucleic acid (e.g., mRNA or genomic DNA) is evaluated, wherein the level, e.g., the presence or absence, of SEF protein or nucleic acid is diagnostic for a subject having or at risk of developing a disease or disorder associated with aberrant or unwanted SEF expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted SEF expression or activity. For example, such methods can be used to determine whether a subject can be effectively treated with an agent for breast cancer.

The methods of the invention can also be used to detect genetic alterations in a SEF gene, thereby determining if a subject with the altered gene is at risk for a disorder characterized by aberrant regulation in SEF protein activity or nucleic acid expression, such as a cellular proliferative disorder. In preferred embodiments, the methods include detecting, in a sample from the subject, the presence or absence of a genetic alteration characterized by at least one of an alteration affecting the integrity of a gene encoding a SEF-protein, or the mis-expression of the SEF gene. For example, such genetic alterations can be detected by ascertaining the existence of at least one of 1) a deletion of one or more nucleotides from a SEF gene; 2) an addition of one or more nucleotides to a SEF gene; 3) a substitution of one or more nucleotides of a SEF gene, 4) a chromosomal rearrangement of a SEF gene; 5) an alteration in the level of a messenger RNA transcript of a SEF gene, 6) aberrant modification of a SEF gene, such as of the methylation pattern of the genomic DNA, 7) allelic loss of a SEF gene, and 8) inappropriate post-translational modification of a SEF-protein.

An alteration can be detected without a probe/primer in a polymerase chain reaction, such as anchor PCR or RACE PCR, or, alternatively, in a ligation chain reaction (LCR), the latter of which can be particularly useful for detecting point mutations in the SEF-gene. This method can include the steps of collecting a sample of cells from a subject, isolating nucleic acid (e.g., genomic, mRNA or both) from the sample, contacting the nucleic acid sample with one or more primers which specifically hybridize to a SEF gene under conditions such that hybridization and amplification of the SEF gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein. Alternatively, other amplification methods described herein or known in the art can be used.

In another embodiment, mutations in a SEF gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in SEF can be identified by hybridizing a sample and control nucleic acids, e.g., DNA or RNA, two dimensional arrays, e.g., chip based arrays. Such arrays include a plurality of addresses, each of which is positionally distinguishable from the other. A different probe is located at each address of the plurality. The arrays can have a high density of addresses, e.g., can contain hundreds or thousands of oligonucleotides probes (Cronin et al. (1996) Human Mutation 7: 244-255; Kozal et al. (1996) Nature Medicine 2: 753-759). For example, genetic mutations in SEF can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the SEF gene and detect mutations by comparing the sequence of the sample SEF with a corresponding control sequence. Automated sequencing procedures can be utilized when performing the diagnostic assays (Naeve et al. (1995) Biotechniques 19:448-53), including sequencing by mass spectrometry.

Other methods for detecting mutations in the SEF gene include methods in which protection from cleavage agents is used to detect mismatched bases in RNA/RNA or RNA/DNA heteroduplexes (Myers et al. (1985) Science 230:1242; Cotton et al. (1988) Proc. Natl. Acad Sci USA 85:4397; Saleeba et al. (1992) Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in SEF cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In other embodiments, alterations in electrophoretic mobility will be used to identify mutations in SEF genes. For example, single strand conformation polymorphism (SSCP) can be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control SEF nucleic acids will be denatured and allowed to renature. The secondary structure of single-stranded nucleic acids varies according to sequence, the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension (Saiki et al. (1986) Nature 324:163); Saiki et al. (1989) Proc. Natl. Acad. Sci USA 86:6230).

In another embodiment, allele specific amplification technology which depends on selective PCR amplification can be used in conjunction with the instant invention. In a preferred embodiment the allele specific amplification is directed to SEF. Oligonucleotides used as primers for specific amplification can carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6: 1). It is anticipated that in certain embodiments amplification can also be performed using Taq ligase for amplification (Barany (1991) Proc. Natl. Acad. Sci USA 88:189-93). In such cases, ligation will occur only if there is a perfect match at the 3′ end of the 5′ sequence making it possible to detect the presence of a known mutation at a specific site by looking for the presence or absence of amplification.

The methods described herein can be performed, for example, by utilizing pre-packaged diagnostic kits comprising at least one probe nucleic acid or antibody reagent described herein, which can be conveniently used, e.g., in clinical settings to diagnose patients exhibiting symptoms or family history of a disease or illness involving a SEF gene.

In other preferred embodiments, the diagnosis and prognostic methods described herein detect an individual that has a SEF mediated disorder or is at risk of developing a SEF mediated disorder. In one embodiment, the invention comprises detecting or measuring expression of a sef allele in said individual or in a sample obtained from said individual, and comparing the detected or measured expression with a suitable control, wherein increased or decreased expression relative to said control is indicative that the individual has or is at risk of developing a SEF mediated disorder, and wherein said sef allele encodes a polypeptide having at least about 90% amino acid sequence identity with SEQ ID NO:2 and comprises an arginine at a position corresponding to position 100 of SEQ ID NO:2, an alanine at a position corresponding to position 36 of SEQ ID NO:2, a glutamic acid at a position corresponding to position 248 of SEQ ID NO:2, a methionine at a position corresponding to position 255 of SEQ ID NO:2, a valine at a position corresponding to position 301 of SEQ ID NO:2, and/or an alanine at a position corresponding to position 608 of SEQ ID NO:2.

In particularly preferred embodiments, the diagnostic and prognostic methods described herein detect a protein of SEQ ID NO:2, or a nucleic acid encoding that protein (e.g., SEQ ID NO:1)

Use of SEF Molecules as Surrogate Markers

The SEF molecules of the invention are also useful as markers of disorders or disease states, as markers for precursors of disease states, as markers for predisposition of disease states, as markers of drug activity, or as markers of the pharmacogenomic profile of a subject. In a preferred embodiment SEF molecules of the invention are useful markers for cancer (e.g., breast cancer, ovarian cancer, prostate cancer, colon cancer). Using the methods described herein, the presence, absence and/or quantity of the SEF molecules of the invention can be detected, and can be correlated with one or more biological states in vivo. For example, the SEF molecules of the invention can serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers can serve to indicate whether a particular course of treatment is effective in lessening a disease state or disorder (e.g., breast cancer). Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease can be made using cholesterol levels as a surrogate marker, and an analysis of HIV infection can be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The SEF molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker can be indicative of the concentration of the drug in a biological tissue, in that the marker is either expressed or transcribed or not expressed or transcribed in that tissue in relationship to the level of the drug. In this fashion, the distribution or uptake of the drug can be monitored by the pharmacodynamic marker. Similarly, the presence or quantity of the pharmacodynamic marker can be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug can be sufficient to activate multiple rounds of marker (e.g., a SEF marker) transcription or expression, the amplified marker can be in a quantity which is more readily detectable than the drug itself. Also, the marker can be more easily detected due to the nature of the marker itself; for example, using the methods described herein, anti-SEF antibodies can be employed in an immune-based detection system for a SEF protein marker, or SEF-specific radiolabeled probes can be used to detect a SEF mRNA marker. Furthermore, the use of a pharmacodynamic marker can offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am. J. Health-Syst. Pharm. 56 Suppl. 3: S16-S20.

The SEF molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35:1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, can be selected. For example, based on the presence or quantity of RNA, or protein (e.g., SEF protein or RNA) for specific tumor markers in a subject, a drug or course of treatment can be selected that is optimized for the treatment of the specific tumor likely to be present in the subject. Similarly, the presence or absence of a specific sequence mutation in SEF DNA can correlate with a SEF drug response. The use of pharmacogenomic markers therefore permits the application of the most appropriate treatment for each subject without having to administer the therapy.

Transgenic Animals

Transgenic animals, in which the genome of the animal host is altered using recombinant DNA techniques, can be constructed. In one embodiment, the alteration is not heritable (e.g., somatic cells, such as progenitor cells in bone marrow, are altered). In another embodiment, the alteration is heritable (the germ line is altered). Transgenic animals can be constructed using standard techniques or other suitable methods (see e.g., Cooke. M. P. et al., Cell, 65: 281-291 (1991) regarding alteration of T lymphocytes; Hanahan, D., Science, 246: 1265-1275, (1989); Anderson et al., U.S. Pat. No. 5,399,346).

In one aspect, an endogenous mammalian SEF gene can be inactivated or disabled, in whole or in part, in a suitable animal host (e.g., by gene disruption techniques) to produce a transgenic animal. Nucleic acids of the present invention can be used to assess successful construction of a host containing an inactivated or disabled SEF gene (e.g., by Southern hybridization). In addition, successful construction of a host containing an inactivated or disabled SEF gene can be assessed by suitable assays which monitor the function of the encoded SEF protein. Such animals can be used to assess the effect of SEF inactivation on endothelial cell function, cardiovascular disease, and cellular proliferation.

In another embodiment, a nucleic acid encoding a mammalian SEF protein or polypeptide is introduced into a suitable host to produce a transgenic animal. In a preferred embodiment, endogenous SEF genes present in the transgenic animals are inactivated (e.g., simultaneously with introduction of the nucleic acid by homologous recombination, which disrupts and replaces the endogenous gene). For example, a transgenic animal (e.g., a mouse, guinea pig, sheep) capable of expressing a nucleic acid encoding a mammalian SEF protein of a different mammalian species (e.g., a human SEF such as the SEF encoded by SEQ ID NO:1) can be produced, and provides a convenient animal model for assessing the function of the introduced receptor. In addition, a test agent can be administered to the transgenic animal, and the effect of the agent on a SEF activity mediated function (e.g., regulation of FGF signaling) can be monitored as described herein or using other suitable assays. In this manner, agents which inhibit or promote SEF function can be identified or assessed for in vivo effect.

Methods of Therapy

Methods of Treatment:

The present invention provides for both prophylactic and therapeutic methods of treating a subject at risk of (or susceptible to) a disorder or having a disorder associated with aberrant or unwanted SEF expression or activity. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, Ribozymes, antisense oligonucleotides, and RNAi.

With regards to both prophylactic and therapeutic methods of treatment, such treatments can be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the SEF molecules of the present invention or SEF modulators according to that individual's drug response genotype. Pharmacogenomics allows a clinician or physician to target prophylactic or therapeutic treatments to patients who will most benefit from the treatment and not to provide this treatment to patients who will experience toxic drug-related side effects.

In one aspect, the invention provides a method for preventing in a subject, a disease or condition associated with an aberrant or unwanted SEF expression or activity, by administering to the subject a SEF or an agent which modulates SEF expression or at least one SEF activity, e.g., regulation of MAPK phosphorylation, cellular proliferation, FGF signaling, regulation of FiRE activation. In a preferred embodiment the invention provides a method for preventing cancer (e.g., breast cancer). Subjects at risk for a disease which is caused or contributed to by aberrant or unwanted SEF expression or activity can be identified by, for example, any or a combination of diagnostic or prognostic assays as described herein. Administration of a prophylactic agent can occur prior to the manifestation of symptoms characteristic of the SEF aberrance, such that a disease or disorder is prevented or, alternatively, delayed in its progression. Depending on the type of SEF aberrance, for example, a SEF, SEF agonist or SEF antagonist agent can be used for treating the subject. The appropriate agent can be determined based on screening assays for modulating SEF described herein, e.g. an agent which was found to modulate the binding of SEF to FGFR, to modulate the phosphorylation of MAPK, to modulate FGF-induced cell response, e.g. cellular proliferation, or to modulate FiRE activation.

It is possible that some SEF disorders can be caused, at least in part, by an abnormal level of gene product, or by the presence of a gene product exhibiting abnormal activity. As such, the reduction in the level and/or activity of such gene products would bring about the amelioration of disorder symptoms.

The SEF molecules can act as novel diagnostic targets and therapeutic agents for controlling one or more of a cellular proliferative and/or differentiative disorder, e.g., ovarian cancer, breast cancer, colon cancer and prostate cancer, a FGF receptor related disorder, cardiovascular disorder, e.g., arteriosclerosis, coronary artery disease, ischemia reperfusion injury, restenosis, arterial inflammation, hypertension and an endothelial disorder, and a kidney disorder, e.g., glomerulonephritits, vascular nephropathy, renal failure, and glomerular disease.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the term “cancer” (also used interchangeably with the terms, “hyperproliferative” and “neoplastic”) refers to cells having the capacity for autonomous growth, i.e., an abnormal state or condition characterized by rapidly proliferating cell growth. Cancerous disease states may be categorized as pathologic, i.e., characterizing or constituting a disease state, e.g., malignant tumor growth, or may be categorized as non-pathologic, i.e., a deviation from normal but not associated with a disease state, e.g., cell proliferation associated with wound repair. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

The SEF molecules of the invention can be used to monitor, treat and/or diagnose a variety of proliferative disorders. Preferentially the SEF molecules of the invention can be used to monitor, treat and/or diagnose ovarian cancer, breast cancer, colon cancer and prostate cancer. Additional proliferative disorders disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

As used herein, disorders involving the heart, or “cardiovascular disease” or a “cardiovascular disorder” includes a disease or disorder which affects the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. A cardiovascular disorder includes, but is not limited to disorders such as arteriosclerosis, atherosclerosis, cardiac hypertrophy, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, valvular disease, including but not limited to, valvular degeneration caused by calcification, rheumatic heart disease, endocarditis, or complications of artificial valves; atrial fibrillation, long-QT syndrome, congestive heart failure, sinus node dysfunction, angina, heart failure, hypertension, atrial fibrillation, atrial flutter, pericardial disease, including but not limited to, pericardial effusion and pericarditis; cardiomyopathies, e.g., dilated cardiomyopathy or idiopathic cardiomyopathy, myocardial infarction, coronary artery disease, coronary artery spasm, ischemic disease, arrhythmia, sudden cardiac death, and cardiovascular developmental disorders (e.g., arteriovenous malformations, arteriovenous fistulae, raynaud's syndrome, neurogenic thoracic outlet syndrome, causalgia/reflex sympathetic dystrophy, hemangioma, aneurysm, cavernous angioma, aortic valve stenosis, atrial septal defects, atrioventricular canal, coarctation of the aorta, ebsteins anomaly, hypoplastic left heart syndrome, interruption of the aortic arch, mitral valve prolapse, ductus arteriosus, patent foramen ovale, partial anomalous pulmonary venous return, pulmonary atresia with ventricular septal defect, pulmonary atresia without ventricular septal defect, persistance of the fetal circulation, pulmonary valve stenosis, single ventricle, total anomalous pulmonary venous return, transposition of the great vessels, tricuspid atresia, truncus arteriosus, ventricular septal defects). A cardiovascular disease or disorder also can include an endothelial cell disorder.

As used herein, an “endothelial cell disorder” includes a disorder characterized by aberrant, unregulated, or unwanted endothelial cell activity, e.g., proliferation, migration, angiogenesis, or vascularization; or aberrant expression of cell surface adhesion molecules or genes associated with angiogenesis, e.g., TIE-2, FLT and FLK. Endothelial cell disorders include tumorigenesis, tumor metastasis, psoriasis, diabetic retinopathy, endometriosis, Grave's disease, ischemic disease (e.g., atherosclerosis), and chronic inflammatory diseases (e.g., rheumatoid arthritis).

Disorders involving the kidney include, but are not limited to, congenital anomalies including, but not limited to, cystic diseases of the kidney, that include but are not limited to, cystic renal dysplasia, autosomal dominant (adult) polycystic kidney disease, autosomal recessive (childhood) polycystic kidney disease, and cystic diseases of renal medulla, which include, but are not limited to, medullary sponge kidney, and nephronophthisis-uremic medullary cystic disease complex, acquired (dialysis-associated) cystic disease, such as simple cysts; glomerular diseases including pathologies of glomerular injury that include, but are not limited to, in situ immune complex deposition, that includes, but is not limited to, anti-GBM nephritis, Heymann nephritis, and antibodies against planted antigens, circulating immune complex nephritis, antibodies to glomerular cells, cell-mediated immunity in glomerulonephritis, activation of alternative complement pathway, epithelial cell injury, and pathologies involving mediators of glomerular injury including cellular and soluble mediators, acute glomerulonephritis, such as acute proliferative (poststreptococcal, postinfectious) glomerulonephritis, including but not limited to, poststreptococcal glomerulonephritis and nonstreptococcal acute glomerulonephritis, rapidly progressive (crescentic) glomerulonephritis, nephrotic syndrome, membranous glomerulonephritis (membranous nephropathy), minimal change disease (lipoid nephrosis), focal segmental glomerulosclerosis, membranoproliferative glomerulonephritis, IgA nephropathy (Berger disease), focal proliferative and necrotizing glomerulonephritis (focal glomerulonephritis), hereditary nephritis, including but not limited to, Alport syndrome and thin membrane disease (benign familial hematuria), chronic glomerulonephritis, glomerular lesions associated with systemic disease, including but not limited to, systemic lupus erythematosus, Henoch-Schönlein purpura, bacterial endocarditis, diabetic glomerulosclerosis, amyloidosis, fibrillary and immunotactoid glomerulonephritis, and other systemic disorders; diseases affecting tubules and interstitium, including acute tubular necrosis and tubulointerstitial nephritis, including but not limited to, pyelonephritis and urinary tract infection, acute pyelonephritis, chronic pyelonephritis and reflux nephropathy, and tubulointerstitial nephritis induced by drugs and toxins, including but not limited to, acute drug-induced interstitial nephritis, analgesic abuse nephropathy, nephropathy associated with nonsteroidal anti-inflammatory drugs, and other tubulointerstitial diseases including, but not limited to, urate nephropathy, hypercalcemia and nephrocalcinosis, and multiple myeloma; diseases of blood vessels including benign nephrosclerosis, malignant hypertension and accelerated nephrosclerosis, renal artery stenosis, and thrombotic microangiopathies including, but not limited to, classic (childhood) hemolytic-uremic syndrome, adult hemolytic-uremic syndrome/thrombotic thrombocytopenic purpura, idiopathic HUS/TTP, and other vascular disorders including, but not limited to, atherosclerotic ischemic renal disease, atheroembolic renal disease, sickle cell disease nephropathy, diffuse cortical necrosis, and renal infarcts; urinary tract obstruction (obstructive uropathy); urolithiasis (renal calculi, stones); and tumors of the kidney including, but not limited to, benign tumors, such as renal papillary adenoma, renal fibroma or hamartoma (renomedullary interstitial cell tumor), angiomyolipoma, and oncocytoma, and malignant tumors, including renal cell carcinoma (hypernephroma, adenocarcinoma of kidney), which includes urothelial carcinomas of renal pelvis.

An FGF related disorder is a disorder caused by aberrant FGFR signaling. Such disorders include cellular proliferative disorders, cardiovascular disorders, and kidney disorders as described herein.

As discussed, successful treatment of SEF disorders can be brought about by techniques that serve to inhibit the expression or activity of target gene products. For example, compounds, e.g., an agent identified using an assays described above, that proves to exhibit negative modulatory activity, can be used in accordance with the invention to prevent and/or ameliorate symptoms of SEF disorders. Such molecules can include, but are not limited to peptides, phosphopeptides, small organic or inorganic molecules, or antibodies (including, for example, polyclonal, monoclonal, humanized, human, anti-idiotypic, chimeric or single chain antibodies, and Fab, Fab′FV, F(ab′)₂ and Fab expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity. Antisense, ribozyme and RNAi molecules are discussed above.

It is possible that the use of antisense, ribozyme, and/or RNAi molecules to reduce or inhibit mutant gene expression can also reduce or inhibit the translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Another method by which nucleic acid molecules can be utilized in treating or preventing a disease characterized by SEF expression is through the use of aptamer molecules specific for SEF protein. Aptamers are nucleic acid molecules having a tertiary structure which permits them to specifically or selectively bind to protein ligands (see, e.g., Osborne et al. (1997) Curr. Opin. Chem Biol. 1: 5-9; and Patel (1997) Curr Opin Chem Biol 1:32-46). Since nucleic acid molecules can in many cases be more conveniently introduced into target cells than therapeutic protein molecules can be, aptamers offer a method by which SEF protein activity can be specifically decreased without the introduction of drugs or other molecules which can have pluripotent effects.

The identified compounds that inhibit target gene expression, synthesis and/or activity can be administered to a patient in a therapeutically effective amount to prevent, treat or ameliorate SEF disorders. A therapeutically effective amount refers to that amount of the compound sufficient to result in amelioration of symptoms of the disorders. Toxicity and therapeutic efficacy of such compounds can be determined by standard pharmaceutical procedures as described above.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine uSEFul doses in humans. Levels in plasma can be measured, for example, by high performance liquid chromatography.

Another example of determination of effective dose for an individual is the ability to directly assay levels of “free” and “bound” compound in the serum of the test subject. Such assays can utilize antibody mimics and/or “biosensors” that have been created through molecular imprinting techniques. The compound which is able to modulate SEF activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell et al (1996) Current Opinion in Biotechnology 7:89-94 and in Shea (1994) Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis et al (1993) Nature 361:645-647. Through the use of isotope-labeling, the “free” concentration of compound which modulates the expression or activity of SEF can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. A rudimentary example of such a “biosensor” is discussed in Kriz et al (1995) Analytical Chemistry 67:2142-2144.

Another aspect of the invention pertains to methods of modulating SEF expression or activity for therapeutic purposes. Accordingly, in an exemplary embodiment, the modulatory method of the invention involves contacting a cell with a SEF or agent that modulates one or more of the activities of SEF protein activity associated with the cell. An agent that modulates SEF protein activity can be an agent as described herein, such as a nucleic acid or a protein, a naturally-occurring target molecule of a SEF protein (e.g., a SEF substrate or receptor), a SEF antibody, a SEF agonist or antagonist, a peptidomimetic of a SEF agonist or antagonist, or other small molecule.

In one embodiment, the agent stimulates one or more SEF activities. Examples of such stimulatory agents include active SEF protein and a nucleic acid molecule encoding SEF. Examples of such inhibitory agents include antisense SEF nucleic acid molecules, anti-SEF antibodies, and SEF inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a SEF protein or nucleic acid molecule. In one embodiment, the method involves administering an agent (e.g., an agent identified by a screening assay described herein), or combination of agents that modulates (e.g., up regulates or down regulates) SEF expression or activity. In another embodiment, the method involves administering a SEF protein or nucleic acid molecule as therapy to compensate for reduced, aberrant, or unwanted SEF expression or activity.

Stimulation of SEF activity is desirable in situations in which SEF is abnormally downregulated and/or in which increased SEF activity SEF likely to have a beneficial effect. For example, stimulation of SEF activity is desirable in situations in which a SEF is downregulated and/or in which increased SEF activity is likely to have a beneficial effect, e.g. cellular proliferation. Likewise, inhibition of SEF activity is desirable in situations in which SEF is abnormally upregulated and/or in which decreased SEF activity is likely to have a beneficial effect.

Modes of Administration

According to the method, one or more agents can be administered to the host by an appropriate route, either alone or in combination with another drug. A therapeutically effective amount of an agent (e.g., a SEF peptide which inhibits SEF function, an anti-SEF antibody or antigen-binding fragment thereof) is administered. An therapeutically effective amount is an amount sufficient to achieve the desired therapeutic or prophylactic effect, under the conditions of administration, such as an amount sufficient for inhibition or promotion of SEF function, and thereby, inhibition or promotion, respectively, of a SEF-mediated process (e.g., a cellular proliferative response).

A variety of routes of administration are possible including, but not necessarily limited to oral, dietary, topical, transdermal, parenteral (e.g., intravenous, intraarterial, intramuscular, subcutaneous injection), and inhalation (e.g., intrabronchial, intranasal or oral inhalation, intranasal drops) routes of administration, depending on the agent and disease or condition to be treated. Formulation of an agent to be administered will vary according to the route of administration selected (e.g., solution, emulsion, capsule). An appropriate composition comprising the agent to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils, for instance. Intravenous vehicles can include various additives, preservatives, or fluid, nutrient or electrolyte replenishers and the like (See, generally, Remington's Pharmaceutical Sciences, 17th Edition, Mack Publishing Co., PA, 1985). For inhalation, the agent can be solubilized and loaded into a suitable dispenser for administration (e.g., an atomizer, nebulizer or pressurized aerosol dispenser).

Furthermore, where the agent is a protein or peptide, the agent can be administered via in vivo expression of the recombinant protein. In vivo expression can be accomplished via somatic cell expression according to suitable methods (see, e.g. U.S. Pat. No. 5,399,346). In this embodiment, nucleic acid encoding the protein can be incorporated into a retroviral, adenoviral or other suitable vector (preferably, a replication deficient infectious vector) for delivery, or can be introduced into a transfected or transformed host cell capable of expressing the protein for delivery. In the latter embodiment, the cells can be implanted (alone or in a barrier device), injected or otherwise introduced in an amount effective to express the protein in a therapeutically effective amount.

### EXEMPLIFICATION

### Example 1

### Cloning of Human SEF

Full-length human SEF was cloned by first searching databases (National Center for Biotechnology information (NCBI), Bethesda, Md.) for sequences similar to the interleukin 17 receptor coding region using the BlastX algorithm (Altschul et al., J. Mol. Biol. 215: 403-410, 1990). Based on gene prediction the entire open-reading frame of SEF was amplified by PCR via PLATINUM Taq DNA polymerase (thermostable DNA polymerase, BD Biosciences, Palo Alto, Calif.) from a mixture of human cDNA libraries (Clontech division of BD Biosciences, Palo Alto, Calif.).

### Example 2

### Tissue Expression Pattern of SEF

SEF was represented as an oligonucleotide probe on custom oligonucleotide microarrays, together with all of the non-redundant human genes from the public databases. To accomplish this, a 50,000-element screening DNA microarray (consisting of two ˜25 k arrays) was designed. For those assemblies where gene orientation was not clear (with respect to transcription), four 60mer probes were designed and represented as an oligonucleotide probe, two for each strand. The remaining sequences (of known orientation) were represented as an oligonucleotide probe by a single unique 60mer probe. The rest of the 50 k microarray was populated by single 60mer probes for ˜5,000 non-redundant RefSeq human full-length genes and ˜13,000 non-redundant Unigene EST clusters derived from EC, heart, aorta or brain libraries. This custom 50 k screening microarray thus contained ˜32,000 unique genes and, likely represents the majority of all expressed human genes as revealed by publicly available sequences of the human genome and large-scale EST sequencing projects.

Competitive hybridizations were performed on cell lines and tissue samples. RNA was isolated from the following cells and cell lines: cultured HUVECs (under standard cultured conditions, or individually treated with several separate stimuli including shear stress, IL-1β, TNFα, TGFβ or VEGF), skin fibroblasts, lung fibroblasts, aortic smooth muscle, peripheral blood leukocytes, monocytes, renal mesangial cells, astroglioma 172, HepG2 (HB-8065, ATCC, Manassas, Va.), platelet-PH2, platelet-7A, platelet-7B drug treated, (PLAVIX, antiplatelet drug, Bristol-Myers Squibb, New York, N.Y.), platelet 6B drug treated, (PLAVIX, antiplatelet drug, Bristol-Myers Squibb, New York, N.Y.) (Table 1).

Additionally, RNA was also isolated from the following tissues, adipose, skeletal muscle, stomach, ventricle, atrium, salivary gland, adrenal, eye, liver, lung, spleen, thymus, bladder, ileum, transverse colon, pancreas, gonad (both testis and ovary), bone marrow, brain cortex, brain stem, brain cerebellum, lymph nodes, and kidney (Table 2). Extracted total RNA was first amplified using in vitro transcription protocols, and equivalent amounts of each of these synthesized RNAs were mixed to give rise to a common reference pool. In one set of experiments, each individually transcribed RNA (labeled with the Cy5 fluorescent label, Cy5 and Cy3 are fluorescent labels widely used in microarray probe labeling) was competitively hybridized for 48 hours to the 50K microarray against the common reference pool (labeled with the Cy3 fluorescent label). A second set of experiments was repeated under the same hybridization conditions with fluorescent reversed labeling of the individual samples (Cy3) and the common reference pool (Cy5). The combined data were analyzed utilizing strictly defined statistical methods (Rosetta Inpharmatics built “platform specific error model”, p<0.05) and the RESOLVER package of software tools (Rosetta Biosoftware, Kirkland, Wash.).

Results: The results demonstrated SEF expression in HUVECs treated with IL-1β, TNF-α, and TGF-α, as well as angiogenic HUVECs, aortic smooth muscle, skin fibroblasts, adipose, skeletal muscle, ventricle, atrium, adrenal, eye, bladder, gonads, brain stem and kidney.

TABLE 1

| CONDITIONS | EXPRESSION |
|---|---|
| HUVEC, static | −0.0443 |
| HUVEC, shear | −0.5216 |
| HUVEC, IL- | 0.2974 |
| 1 beta | |
| HUVEC, | 0.1961 |
| TNFalpha | |
| HUVEC, | 0.1724 |
| TGFbeta | |
| HUVEC, | 0.788 |
| angiogenic | |
| aortic smooth | 0.1663 |
| muscle | |
| skin fibroblasts | 5.5724 |
| lung fibroblasts | −0.2092 |
| PBL/buffy coat | −0.8419 |
| monocytes | −0.5091 |
| renal mesangial | −0.6447 |
| cells | |
| astroglioma 172 | −0.3712 |
| HepG2 | −0.328 |
| platelet-PH2 | −0.551 |
| platelet-7A | −0.623 |
| platelet 7B, drug | −0.5515 |
| treated | |
| platelet 6B, drug | −0.5982 |
| treated | |

TABLE 2

| TISSUE | EXPRESSION |
|---|---|
| adipose | 0.7944 |
| sk.Muscle | 0.0522 |
| stomach | −0.3208 |
| ventricle(L) | 0.748 |
| atrium(R) | 0.545 |
| salivarygland | 0.1136 |
| adrenal | 2.4524 |
| eye | 0.706 |
| liver | −0.0938 |
| lung | −0.7416 |
| spleen | 0.1317 |
| thymus | −0.6898 |
| bladder | 0.1762 |
| ileum | −1.2723 |
| transversecolon | −0.5741 |
| pancreas | 0.1109 |
| gonad | 1.4218 |
| bonemarrow | −0.2059 |
| braincortex | 0.1411 |
| brainstem | 0.6383 |
| braincerebellum | −0.246 |
| lymphnodes | −0.1422 |
| kidney | 1.8562 |

### Example 3

### SEF is Expressed in HUVECs

RT-PCR analysis was conducted on untreated and treated HUVECs (IL-1 treated (16 hrs), TNF-α treated (16 hrs or 24 hrs), platelets, lung fibroblast, skin fibroblast, and aortic smooth muscle cells. Briefly, RNA was extracted and reverse transcribed using SUPERSCRIPT II reverse transcriptase (Invitrogen Life Technologies, Carlsbad, Calif.) according to manufacturers' instructions. The cDNA was subjected to PCR via PLATINUM Taq DNA polymerase (thermostable DNA polymerase, BD Biosciences, Palo Alto, Calif.) according to manufacturer's instruction. The amplified PCR product was visualized following agarose gel electrophoresis.

Results: The results indicated that SEF is expressed in untreated HUVECs, IL-1β treated HUVECs, TNF-α treated HUVECs, lung fibroblast, skin fibroblast and aortic smooth muscle. SEF expression was not observed in platelets.

Materials and Methods

The following materials and methods were used in the work described in Examples 4-10.

SEF Truncation Constructs

Expression constructs were synthesized by amplifying the open-reading frame or portions of SEF with PLATINUM Taq DNA polymerase (thermostable DNA polymerase, BD Biosciences, Palo Alto, Calif.) and inserting the product into the PFLAG-CMV-1 expression vector (Sigma, St. Louis, Mo.), PGEMT-EZ expression vector (Promega, Madison, Wis.), and PCDNA4 version A expression plasmid (Invitrogen Life Technologies, Carlsbad, Calif.).

The following constructs were prepared as FLAG tagged proteins in PFLAG-CMV-1 expression vector (Sigma-Aldrich, St. Louis, Mo.): SEF-FL contained the entire open reading frame of SEF (amino acids 36-739 of SEQ ID NO: 2) and was cloned with primers as described in SEQ ID NO:5 and SEQ ID NO:6. SEF-ICD-TM contained amino acids 284-739 of SEQ ID NO: 2 which forms the intracellular and transmembrane domains of SEF and was cloned with the primers described in SEQ ID NO: 9 and SEQ ID NO:10. SEF-ECD-TM contained amino acids 36-353 of SEQ ID NO: 2 which forms the extracellular and transmembrane domains of SEF and was cloned with the primers described in SEQ ID NO:7 and SEQ ID NO:8, and SEF-noSH3 which contained amino acids 36-510 of SEQ ID NO: 2 encoding the extracellular, transmembrane and partial intracellular domains of SEF and lacked the SH3-interacting region. SEF-noSH3 was cloned with the primers described in SEQ ID NO:11 and SEQ ID NO:12. Similarly, the following constructs were prepared as Myc tagged proteins in PCDNA4 VERSION A expression plasmid (Invitrogen Life Technologies, Carlsbad, Calif.): SEF-FL contained the entire open reading frame of SEF (amino acids 1-739 of SEQ ID NO: 2). SEF-ICD-TM contained amino acids 284-739 of SEQ ID NO: 2 which forms the intracellular and transmembrane domains of SEF, SEF-ECD-TM contained amino acids 1-353 of SEQ ID NO: which forms the extracellular and transmembrane domains of SEF, and SEF-noSH3 which contained amino acids 36-510 of SEQ ID NO: 2 encoding the extracellular, transmembrane and partial intracellular domains of SEF and lacked the SH3-interacting region.

Cell Culture and Transfection—Human embryonic kidney 293T cells were maintained in DULBECCO'S MODIFIED EAGLE MEDIUM (DMEM, GIBCO brand cell culture products available from Invitrogen Life Technologies, Carlsbad, Calif.) supplemented with 10% fetal bovine serum (FBS), 100 μg/ml penicillin and 100 μg/ml streptomycin. Cells were seeded in 6-well plates and cultured overnight. Transfections were performed using FUGENE 6 multi-component lipid-based transfection reagent (Roche Molecular Biochemicals, Indianapolis, Ind.). The total amount of DNA was kept constant in all transfections by supplementing the transfection mixture with empty vector DNA when needed.

Cell lysis and Western blots—Transfected cells were washed once with PBS and lysed for 15 min on ice in 0.5 ml of lysis buffer (20 mM Tris-HCl, pH 7.5, 150 mM NaCl, 1 mM Ethylenediaminetetraacetic acid (EDTA), 1 mM Ethylenebis(oxyethylenenitrilo) tetraacetic acid (EGTA), 1% TRITON X-100 (t-Octylphenoxypolyethoxy ethanol, nonionic detergent, Union Carbide subsidiary of Dow Chemical Co., Midland Mich., obtained from Sigma-Aldrich, St. Louis, Mo.), 25 mM sodium pyrophosphate, 1 mM β-glycerophosphate, 1 mM Na₃VO₄, 1 μg/ml leupeptin). Lysates were clarified by centrifugation at 4° C. for 15 min at 10,000×g. Lysates were boiled in Laemmli sample buffer, fractionated by SDS-PAGE, and transferred to polyvinylidene difluoride membranes. The membranes were blocked with PBS (pH 7.5) containing 0.1% gelatin and 0.05% TWEEN-20 (Polyethylene glycol sorbitan monolaurate, Uniqema, a business unit of ICI Americas Inc., Newcastle, Del., obtained from Sigma-Aldrich, St. Louis, Mo.) and were blotted with the indicated antibody for 2 hrs at room temperature. Blots were then washed twice and incubated with HRP-conjugated goat anti-mouse IgG (Jackson ImmunoResearch Laboratories, West Grove, Pa.) for 1 hr. After washing the membranes, the reactive bands were visualized with ECL+PLUS western blotting detection agents (Amersham Biosciences, Piscataway, N.J.).

Immunoprecipitations—Cells lysates were incubated with 1 μg of indicated antibody and 20 μl of 50% (v/v) protein A-agarose (Pierce, Rockford, Ill.) for 2 hrs with gentle rocking. After three washes with lysis buffer, precipitated complexes were solubilized by boiling in Laemmli sample buffer, fractionated by SDS-PAGE, and transferred to polyvinylidene difluoride membranes and blotted.

### Example 4

### SEF Effects on MAPK and MEK Phosphorylation

Cells were transfected with the FLAG tagged SEF constructs (SEF-FL, SEF-ECD-TM, SEF-ICD-TM, and SEF-noSH3) as described above and cultured for 24 hours. FGF was added at 50 ng/ml for 10 minutes. Transfected cells were washed once with PBS and lysed. Lysates were separated by SDS-PAGE and transferred to polyvinylidene difuoride membranes as described above. The membranes were blotted with antibodies specific for phosphorylated MAPK (Cell Signaling Technology, Beverly, Mass.) or total MAPK (Cell Signaling Technology, Beverly, Mass.) for 2 hrs. After two washes, the blots were incubated with HRP-conjugated goat anti-mouse IgG (Jackson ImmunoResearch Laboratories, West Grove, Pa.) for 1 hr. After washing the membranes, the reactive bands were visualized with the ECL+PLUS western blotting detection agents (Amersham Biosciences, Piscataway, N.J.). Results were measured as the fold of phosphorylation over relative basal level.

In order to determine whether SEF affects MEK phosphorylation, human embryonic kidney 293T cells were transfected with the FUGENE 6 multi-component lipid-based transfection reagent (Roche Molecular Biochemicals, Indianapolis, Ind.) with the FLAG tagged SEF-FL construct or empty vector as described above. Twenty four hours later FGF was added at 50 ng/ml for 10 minutes. Lysates were prepared separated by SDS-PAGE, and transferred to polyvinyldene diflouride membranes and blotted with antibodies specific for phosphorylated MEK (Cell Signaling Technologies, Beverly, Mass.) OR MEK (Cell signaling Technologies, Beverly, Mass.).

Results—Results indicate that SEF inhibited FGF-induced MAPK phosphorylation. Furthermore results indicated that the intracellular domain and SH3 domain of SEF are required for full SEF mediated MAPK inhibition (Table 3). Results also indicated that SEF does not affect MEK phosphorylation, results not shown.

TABLE 3
MAPK PHOSPHORYLATION

| | MAPK-P | |
|---|---|---|
| CONSTRUCTS | (FOLD) | FGF |
| VECTOR | 1.0 | − |
| VECTOR | 5.45 | + |
| SEF-FL | 2.21 | + |
| SEF-ECD-TM | 4.6 | + |
| SEF-ICD-TM | 2.0 | + |
| SEF-no-SH3 | 5.44 | + |

### Example 5

### SEF Interacts with FGFR

293 T cells were cultured and transfected as described above. Briefly 293T cells were transfected with the FGF receptor and one of the following constructs: SEF-FL, SEF-ICD-TM, SEF-ECD-TM, or SEF-noSH3 in the MYC tagged PCDNA4 VERSION A expression plasmid (Invitrogen Life Technologies, Carlsbad, Calif.) as described above. Transfected cells were cultured for twenty-four hours, then either left untreated or were treated with FGF (50 ng/ml) for 10 minutes. Cells were lysed and immunoprecipitated with antibody specific for the MYC tag, as described above. The precipates were separated by SDS-PAGE, transferred, blotted with antibody to the FGF receptor and visualized as described.

Results: Results indicated that SEF forms an immunoprecipitable complex with the FGF receptor both in the presence and absence of FGF. Coimmunoprecipitation of SEF and FGFR1 indicates that these two cell surface proteins can form a stable complex. This data also suggest that SEF may participate in the FGFR signaling pathway.

### Example 6

### SEF Dimer Formation

293T cells were cultured and transfected with full length SEF in MYC tagged PCDNA4 VERSION A, SEF-FL (Invitrogen Life Technologies, Carlsbad, Calif.) and FLAG tagged PFLAG-CMV-1, SEF-FL, (Sigma-Aldrich, St. Louis, Mo.), expression vectors. After transfection the cells were cultured for 24 hours and then lysed. Lysates were incubated with either 1 μg of the anti-FLAG M2 antibody (Sigma-Aldrich, St. Louis, Mo.) or 1 ug of anti-MYC (COVANCE, Princeton, USA) and immunoprecipitated, fractionated, and blotted as described above. Membranes were blotted with anti-FLAG M2 antibody (Sigma-Aldrich, St. Louis, Mo.) or anti-MYC antibodies. After two washes, the blots were incubated with HRP-conjugated goat anti-mouse IgG (Jackson ImmunoResearch Laboratories, West Grove, Pa.) for 1 hr. After washing the membranes, the reactive bands were visualized with ECL+PLUS western blotting detection agents (Amersham Biosciences, Piscataway, N.J.).

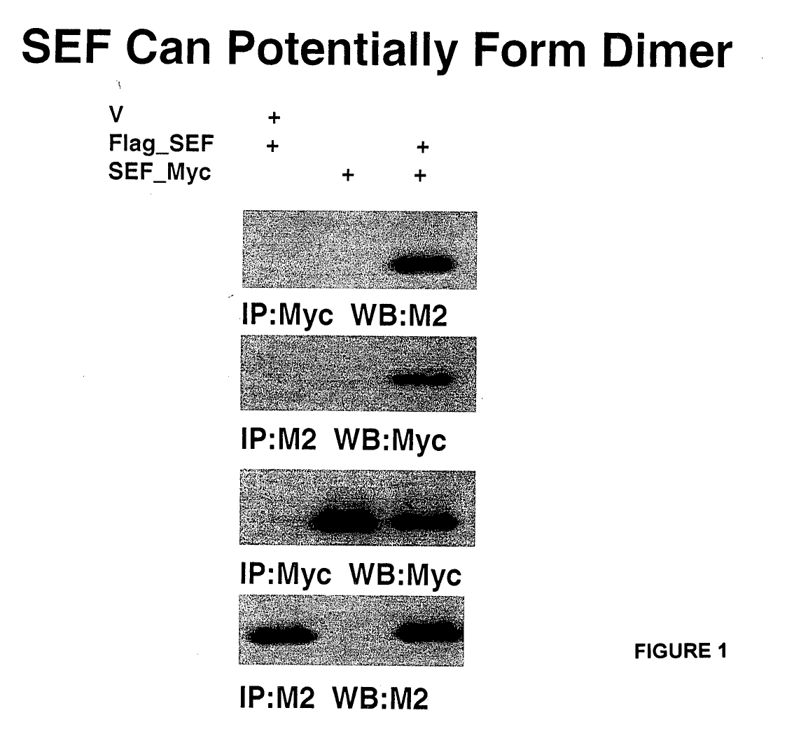

Results: Results indicated that SEF forms a multiunit complex with other SEF molecules (FIG. 1). Lysates from cells transfected with both FLAG tagged SEF and MYC tagged SEF were immunoprecipitated with anti-MYC and then blotted with anti-FLAG M2. Blotting revealed that the anti-MYC immunoprecipitate contained the FLAG tagged SEF construct indicating that SEF forms a complex containing two or more SEF molecules. Similarly, the anti-FLAG M2 immunoprecipitate, from cells transfected with FLAG tagged and MYC tagged SEF, and contained both FLAG tagged SEF and MYC tagged SEF.

### Example 7

### Dose-Dependent Inhibitory Effect of SEF on FGF Induced FiRE Activation

Human embryonic kidney 293T cells were transfected with SEF-FL in the PGEMT-EZ expression vector (Promega, Madison, Wis.), together with 0.5 ug of a FIRE reporter plasmid PGL3-FIRE (Promega, Madison, Wis.) and 0.05 ug of the Renilla luciferase reporter (Promega, Madison, Wis.), as an internal control. Transfected cells were cultured for 20 hours and were either left untreated or treated with FGF (50 ng/ml) for 6 hours. Luciferase activity was measured according to the DUAL-LUCIFERASE reporter assay system (Promega, Madison, Wis.). Results were expressed as relative luciferase activity, by dividing firefly luciferase activity by the Renilla luciferase control activity.

Results: Results indicated that SEF expression inhibited the activation of the FIRE-FGF induced Response Element (Table 4). Furthermore inhibition by full length SEF (SEF-FL) was shown to occur in a dose dependent fashion, with roughly four fold inhibition of FIRE-FGF induced response element in cells transfected with 0.75 ug of SEF-FL (when compared to vector control). Expression of full length SEF resulted in the greatest inhibition of FIRE in this assay. Expression of truncated forms of SEF also inhibited FIRE under the conditions tested.

TABLE 4
EFFECT OF SEF ON FIRE INDUCED
RESPONSE ELEMENT

| | Fold | |
|---|---|---|
| Construct | increase | Std Dev |
| V | 302% | 32% |
| SEF 0.25 uG | 165% | 21% |
| SEF 0.5 uG | 135% | 15% |
| SEF 0.75 uG | 125% | 8% |
| V | 288% | 34% |
| SEF | 120% | 10% |
| SEF_ECD_TM | 217% | 30% |
| SEF_ICD_TM | 157% | 26% |
| SEF_noSH3 | 163% | 44% |

### Example 8

### Generation of SEF Stable Cell Lines

Stable cell lines were generated as follows. A DNA fragment containing FLAG-tagged full length SEF was cloned into PCDNA4.1 VERSION A expression plasmid (Invitrogen Life Technologies, Carlsbad, Calif.). The resulting construct was transfected into NIH3T3 cells (CRL-1658, ATCC, Manassas, Va.) using the FUGENE 6 multi-component lipid-based transfection reagent (Roche Molecular Biochemicals, Indianapolis, Ind.) according to the manufacturer's directions. Cells that had taken up the DNA were selected with 400 ug/ml of Zeocin (Invitrogen Life Technologies, Carlsbad, Calif.). Single colonies were picked 2 weeks after selection and grown. Colonies were picked and analyzed by western blotting to confirm SEF expression.

Positive clones were further characterized by western blot by measuring SEF, MAPK phosphorylation, and total MAPK after treatment with FGF at 10 ng/ml or 50 ng/ml, as described above.

### Example 9

### In Situ Histochemistry (ISH) SEF Expression in Tissue Samples and Breast Tumor Cell Lines

In Situ Hybridization—The tissue preparation and in situ hybidizations were performed by conventional procedures as described in Komuves et al. J. Invest. Dermatol. 115: 353-360, Komuves et al. J. Histochem. Cytochem. 48: 821-830, and Stelnicki et al. J. Invest. Dermatol. 110:110. Following the manufacturer's protocol, digoxigenin-labeled antisense and sense riboprobes were synthesized from SEF DNA templates using reagents purchased from Roche Molecular Biochemicals (Mannheim, Germany). Sectioning, pretreatment of the sections and hybridization of the probes were done under strict RNAase-free conditions. All reagents were prepared using diethyl-pirocarbonate-treated distilled water (DEPC-dH₂O). 15 μm thick sections were collected on positively charged slides and dried at 55° C. overnight. The sections were deparaffinized and rehydrated in HISTOSOLVE (ThermoShandon, Pittsburgh, Pa.) and ethanol and rinsed in DEPC-dH₂O. The sections were treated at room temperature with 0.2 N HCl (20 min), 1.5% H₂O₂ (15 min), 0.3% TRITON X-100 (t-Octylphenoxypolyethoxy ethanol, nonionic detergent, Union Carbide subsidiary of Dow Chemical Co., Midland Mich., obtained from Sigma-Aldrich) (15 min) followed by proteinase K treatment, at 37° C. (30 min). The sections then were washed with triethanolamine buffer, followed by acetylation with acetic anhydride. Following prehybridization in 2×SSC, containing 50% formamide at 37° C., (1 h), the sections were air dried at room temperature. Sections were hybridized with the probes diluted in hybridization solution (2×SSC, containing 50% formamide, 10× Denhardt's, 0.001% SDS, 10 mM Tris, pH7.4, 0.005% sodium pyrophosphate and 500 μg/ml yeast tRNA) at 55° C., overnight. Following hybridization, the sections were washed with 4×SSC (2×15 min), and 2×SSC (2×15 min). The sections then were treated with RNAase A at 37° C., for 30 min, followed by stringency washes in 2×SSC at 37° C. (15 min), 0.1×SSC at 42° C. (40 min), and finally in 0.1×SSC at room temperature (2×15 min). The sections were washed with maleate buffer (30 min) and then blocked with 10 mM Tris buffer, pH7.6, containing 500 mM NaCl, 4% BSA, 0.5% cold-water fish skin gelatin, and 0.05% TWEEN 20 (Polyethylene glycol sorbitan monolaurate, Uniqema, a business unit of ICI Americas Inc., Newcastle, Del., obtained from Sigma-Aldrich, St. Louis, Mo.). The sections were then incubated with anti-digoxigenin antibody, conjugated to peroxidase (Roche Molecular Biochemicals, Mannheim, Germany), for 1 h. The signal was amplified using TSA-Plus kit (Perkin-Elmer Life Sciences, Boston, Mass.) and the signal was detected with Vector Blue substrate (Vector Laboratories, Burlingame, Calif.). Following incubation with substrate the sections were dehydrated in ethanol and HISTOSOLVE (ThermoShandon, Pittsburgh, Pa.) and cover slipped. Hybridization with the sense control probe did not result in detectable signal, indicating the specificity of hybridization.

Microscopy—Slides were observed with an Olympus BX50 microscope (Olympus US, Inc., Melville, N.Y.), using DIC illumination (Differential Interference Contrast). The microscope was equipped with a NIKON DXM1200 digital camera (Technical Instruments San Francisco, Burlingame, Calif.). Digitized images (1280×1024 pixel resolution) were acquired using ACT-1 software (Nikon USA, Melville, N.Y.). Images were resized, cropped and assembled using Photoshop v. 6.0 (Adobe Systems, San Jose, Calif.). Apart from equalizing the background intensities, no other digital modifications of the original digital images were carried out.

Results: In situ hybridizations of normal tissue showed SEF expression in normal epithelial cells of the intercalated ducts of the salivary glands (parotid and submandibular glands), and the collecting ducts in the kidney.

In situ hybridizations of breast tumors showed uniform SEF expression in the epithelial cells of well-differentiated ductal carcinomas. Variable expression was found in less differentiated forms of ductal carcinomas, with several tumors negative for SEF expression. Variable SEF expression was also found in fibroadenoma, cribiform carcinoma, and mucus adenocarcinoma.

The in situ hybridizations of breast tumor cell lines indicated that SEF expression correlated with HER2/neu expression. Accordingly, MDA-231 (ATCC No. 45518, Manassas, Va.), which does not express HER2/neu, displayed the weakens SEF expression, MDA-175 (ATCC No. 45516, Manassas, Va.), a moderate HER2/neu expressor, displayed moderate SEF expression, while SKBR-3 cells, a high HER2/neu expressor, displayed the strongest SEF expression.

### Example 10

### SEF Tissue Expression

Total RNA was prepared from various human tissues by RNA STAT-60 single step RNA extraction (TelTest, Inc., Friendswood, Tex.) according to the manufacturer's instructions (TelTest, Inc., Friendswood, Tex.). Each RNA preparation was treated with DNase I (Ambion, Austin, Tex.) at 37° C. for 1 hour. DNAse I treatment was determined to be complete if the sample required at least 38 PCR amplification cycles to reach a threshold level of fluorescence using β-2 microglobulin as an internal amplicon reference. The integrity of the RNA samples following DNase I treatment was confirmed by agarose gel electrophoresis and ethidium bromide staining. After phenol extraction cDNA was prepared from the sample using the SUPERSCRIPT CHOICE SYSTEM (GIBCO brand products available from Invitrogen Life Technologies, Carlsbad, Calif.) following the manufacturer's instructions. A negative control of RNA without reverse transcriptase was mock reverse transcribed for each RNA sample.

Human SEF expression was measured by TAQMAN quantitative PCR (Perkin Elmer Life Sciences Inc., Boston, Mass.) in cDNA prepared from a variety of normal and diseased (e.g., cancerous) human tissues or cell lines.

Probes were designed by PrimerExpress software (PE Applied Biosystems, Foster City, Calif.) based on the sequence of the human SEF gene. Each human SEF gene probe was labeled using FAM (6-carboxyfluorescein), and the P2-microglobulin reference probe was labeled with a different fluorescent dye, VIC. The differential labeling of the target gene and internal reference gene thus enabled measurement in same well. Forward and reverse primers and the probes for both β2-microglobulin and target gene were added to the TAQMAN Universal PCR Master Mix (PE Applied Biosystems, Foster City, Calif.). Although the final concentration of primer and probe could vary, each was internally consistent within a given experiment. A typical experiment contained 200 nM of forward and reverse primers plus 100 nM probe for β-2 microglobulin and 600 nM forward and reverse primers plus 200 nM probe for the target gene. TaqMan matrix experiments were carried out on an ABI PRISM 7700 Sequence Detection System (PE Applied Biosystems, Foster City, Calif.). The thermal cycler conditions were as follows: hold for 2 min at 50° C. and 10 min at 95° C., followed by two-step PCR for 40 cycles of 95° C. for 15 sec followed by 60° C. for 1 min.

The following method was used to quantitatively calculate human SEF gene expression in the various tissues relative to β-2 microglobulin expression in the same tissue. The threshold cycle (Ct) value is defined as the cycle at which a statistically significant increase in fluorescence is detected. A lower Ct value was indicative of a higher mRNA concentration. The Ct value of the human SEF gene was normalized by subtracting the Ct value of the β-2 microglobulin gene to obtain a _{Δ}Ct value using the following formula: _{Δ}Ct=Ct_{human 59914 and 59921}−Ct_{β-2 microglobulin}. Expression was then calibrated against a cDNA sample showing a comparatively low level of expression of the human SEF gene. The _{Δ}Ct value for the calibrator sample was then subtracted from ΔCt for each tissue sample according to the following formula: _{ΔΔ}Ct=_{Δ}Ct-ₛₐₘₚₗₑ−_{Δ}Ct-_{calibrator}. Relative expression was then calculated using the arithmetic formula given by 2^{-ΔΔCt}. Expression of the target human SEF gene in each of the tissues tested was then graphically represented as discussed in more detail below.

The results indicate significant SEF expression in normal artery, normal vein, kidney, and peripheral blood leukocytes. Higher expression was observed in HUVECs, normal spinal cord, normal brain cortex, normal brain hypothalamus, UUI bladder, normal breast, and normal ovary. Furthermore differential SEF expression was found when normal HUVECs were compared to a tumor hemanginoma (lower SEF expression), normal heart compared to heart that underwent congestive heart failure (higher SEF expression), normal breast tissue compared to breast tumor tissue (lower SEF expression), normal ovary compared to ovarian tumors (lower SEF expression), normal prostate compared to prostate tumors (higher SEF expression) and normal colon compared to colon tumors (higher SEF expression).

What is claimed is:

1. An isolated nucleic acid molecule selected from the group consisting of:
a) an isolated nucleic acid comprising the nucleotide sequence of SEQ ID NO:1 or a portion of SEQ ID NO:1 comprising the coding region,
b) an isolated nucleic acid comprising a nucleotide sequence which has at least about 90% nucleotide sequence identity with the nucleotide of SEQ ID NO:1, or the coding region thereof and comprises one or more nucleotides selected from the group consisting of a cytosine at a position corresponding to position 100 of SEQ ID NO:1, a cytosine at a position corresponding to position 107 of SEQ ID NO:1, a guanine at a position corresponding to position 742 of SEQ ID NO:1, a thymine at a position corresponding to position 764 of SEQ ID NO:1, a guanine at a position corresponding to position 901 of SEQ ID NO:1, and a guanine at a position corresponding to position 1822 of SEQ ID NO:1,
c) an isolated nucleic acid comprising the nucleotide sequence which encodes a polypeptide comprising the amino acid sequence of SEQ ID NO:2 or a biologically active mature form, and
d) an isolated nucleic acid encoding a SEF allelic variant, wherein said SEF variant comprises a sequence, an amino acid sequence having at least about 90% amino acid sequence identity with SEQ ID NO:2 and comprises an amino acid selected from the group consisting of an arginine at position 34 of SEQ ID NO:2, an alanine at position 36 of SEQ ID NO:2, a glutamic acid at position 248 of SEQ ID NO:2, a methionine at position 255 of SEQ ID NO:2, a valine at position 301 of SEQ ID NO:2, and an alanine at position 608 of SEQ ID NO:2 of the corresponding position of SEQ ID NO:2.

2. An isolated vector comprising a recombinant nucleic acid selected from the group consisting of:
a) a nucleic acid comprising the nucleotide sequence of SEQ ID NO:1 or a portion of SEQ ID NO:1 comprising the coding region,
b) a nucleic acid comprising a nucleotide sequence which has at least about 90% nucleotide sequence identity with the nucleotide of SEQ ID NO:1, or the coding region thereof and comprises one or more nucleotides selected from the group consisting of a cytosine at a position corresponding to position 100 of SEQ ID NO:1, a cytosine at a position corresponding to position 107 of SEQ ID NO:1, a guanine at a position corresponding to position 742 of SEQ ID NO:1, a thymine at a position corresponding to position 764 of SEQ ID NO:1, a guanine at a position corresponding to position 901 of SEQ ID NO:1, and a guanine at a position corresponding to position 1822 of SEQ ID NO:1,
c) a nucleic acid which encodes a polypeptide comprising the amino acid sequence of SEQ ID NO:2, or a biologically active mature form, and
d) a nucleic acid encoding a SEF allelic variant, wherein said SEF variant comprises a sequence, an amino acid sequence having at least about 90% amino acid sequence identity with SEQ ID NO:2 and comprises an amino acid selected from the group consisting of an arginine at position 34 of SEQ ID NO:2, an alanine at position 36 of SEQ BD NO:2, a glutamic acid at position 248 of SEQ BD NO:2, a methionine at position 255 of SEQ ID NO:2, a valine at position 301 of SEQ ID NO:2, and an alanine at position 608 of SEQ ID NO:2 of the corresponding position of SEQ ID NO:2.

3. A host cell comprising the recombinant nucleic acid of claim 2, wherein said recombinant nucleic acid is operatively linked to an expression control element.

4. An isolated SEF polypeptide selected from the group consisting of:
a) a polypeptide comprising the amino acid sequence of SEQ ID NO:2 or biologically active mature form, and
b) a polypeptide having an amino acid sequence at least about 90% amino acid sequence identity with SEQ ID NO:2 and an arginine at a position corresponding to position 34 of SEQ ID NO:2, an alanine at a position corresponding to position 36 of SEQ ID NO:2, a glutamic acid at a position corresponding to position 248 of SEQ ID NO:2, a methionine at a position corresponding to position 255 of SEQ ID NO:2, a valine at a position corresponding to position 301 of SEQ ID NO:2, or an alanine at a position corresponding to position 608 of SEQ ID NO:2.

5. A fusion protein comprising a SEF moiety, wherein said SEF moiety has an amino acid sequence selected from the group consisting of:
a) an amino acid sequence of SEQ ID NO:2 or biologically active mature form,
b) an amino acid sequence which is at least about 90% amino acid sequence identity with SEQ ID NO:2 and an arginine at a position corresponding to position 34 of SEQ ID NO:2, an alanine at a position corresponding to position 36 of SEQ ID NO:2, a glutamic acid at a position corresponding to position 248 of SEQ ID NO:2, a methionine at a position corresponding to position 255 of SEQ ID NO:2, a valine at a position corresponding to position 301 of SEQ ID NO:2, or an alanine at a position corresponding to position 608 of SEQ ID NO:2, and
c) an amino acid sequence selected from the group consisting of amino acids 36-739 of SEQ ID NO: 2, amino acids 284-739 of SEQ ID NO:2, amino acids 36-353 of SEQ ID NO:2, and amino acids 36-510 of SEQ ID NO:2, linked to a second moiety.

6. The fusion protein of claim 5 wherein the second moiety is selected from the group consisting of an amino acid, oligopeptide, and polypeptide.

7. A method for producing a SEF polypeptide comprising maintaining the host cell of claim 3 under conditions suitable for expression.

8. A method for detecting the presence of a SEF allelic variant allele, in a sample, comprising:
a) combining the sample with a nucleic acid probe which selectively hybridizes to the nucleic acid sequence of SEQ ID NO:1 under high stringency conditions; and
b) detecting whether the nucleic acid probe hybridizes to a nucleic acid in said sample, wherein hybridization is indicative of the presence of a SEF allele.

9. The method of claim 8, wherein the sample is obtained from a subject having or at risk of developing a disorder selected from the group consisting of a kidney disorder, a cellular proliferative disorder, a FGF receptor related disorder, and a cardiovascular disorder.

10. A method for detecting the presence of a SEF polypeptide comprising the amino acid sequence of claim 4, or a fragment thereof, in a sample, comprising:
a) combining the sample with an antibody or antigen binding fragment thereof which specifically binds to a polypeptide having an amino acid sequence of claim 4 and does not bind SEQ ID NO:4; and
b) detecting an antibody or antigen binding fragment in complex with SEF, wherein the presence of a complex is indicative of the presence of SEF in the sample.

11. The method of claim 10, wherein the sample is obtained from a subject with or at risk of developing a disorder selected from the group consisting of a kidney disorder, a cellular proliferative disorder, a FGF receptor related disorder, and a cardiovascular disorder.

12. The method of claim 10 wherein said antibody is a monoclonal antibody.

13. The method of claim 10 wherein said antibody comprises a detectable label.

14. A kit comprising a nucleic acid probe which selectively hybridizes to the nucleic acid sequence of claim 1 under high stringency conditions and instructions for use.

15. A kit comprising an antibody or antigen binding fragment thereof which selectively binds to a SEF polypeptide comprising the amino acid sequence of claim 4 and instructions for use, wherein said antibody does not bind SEQ ID NO:4.

16. A method for identifying an agent which binds to a SEF having the amino acid sequence of claim 4, comprising the steps of:
a) combining a composition comprising a polypeptide comprising the amino acid sequence of claim 4 with a test compound; and
b) detecting binding of the test compound to said SEF,
wherein a modulator is identified when the test compound binds to the polypeptide.

17. A method for identifying a compound which modulates the activity of a SEF having the amino acid sequence of claim 4 comprising the steps of:
a) combining a composition comprising a polypeptide having amino acid sequence of claim 4 with a test compound; and
b) detecting a change in SEF activity,
wherein a change in the activity of the polypeptide is indicative that the test compound modulates the activity of SEF.

18. The method of claim 17, wherein the test compound is selected from the group consisting of a small molecule, an antibody, a nucleic acid, and a polypeptide.

19. The method of claim 17, wherein the SEF activity is selected from the group consisting of:
a) regulation of MAPK phosphorylation,
b) regulation of an FGF induced response element, and
c) regulation of FGF-induced cellular proliferation.

20. A method of modulating the activity of a SEF polypeptide comprising the amino acid sequence of claim 4, comprising contacting said polypeptide with a compound which modulates the activity of the polypeptide.

21. The method of claim 20, wherein the compound is selected from the group consisting of a small molecule, an antibody, a nucleic acid, and a polypeptide.

22. The method of claim 20, wherein the activity is is selected from the group consisting of:
a) regulation of MAPK phosphorylation,
b) regulation of an FGF induced response element, and
c) regulation of FGF-induced cellular proliferation.

23. A method of identifying an individual that has a SEF mediated disorder or is at risk of developing a SEF mediated disorder, comprising
detecting or measuring expression of a SEF allele in said individual or in a sample obtained from said individual, and
comparing the detected or measured expression with a suitable control, wherein increased or decreased expression relative to said control is indicative that the individual has or is at risk of developing a SEF mediated disorder, and
wherein said SEF allele encodes a polypeptide having at least about 90% amino acid sequence identity with SEQ ID NO:2 and comprises an arginine at a position corresponding to position 100 of SEQ ID NO:2, an alanine at a position corresponding to position 36 of SEQ ID NO:2, a glutamic acid at a position corresponding to position 248 of SEQ ID NO:2, a methionine at a position corresponding to position 255 of SEQ ID NO:2, a valine at a position corresponding to position 301 of SEQ ID NO:2, and/or an alanine at a position corresponding to position 608 of SEQ ID NO:2.

24. The method of claim 23, wherein said sample is obtained from a subject with or at risk of developing a disorder selected from the group consisting of a kidney disorder, a cellular proliferative disorder, a FGF receptor related disorder, and a cardiovascular disorder.

25. A method for evaluating the efficacy of a therapy for treating a SEF mediated disorder in a subject, comprising
detecting or measuring expression of a SEF allele in said subject that has received said therapy or in a sample obtained from said subject, and
comparing the detected or measured expression with a suitable control, wherein increased or decreased expression relative to said control is indicative of the efficacy of said therapy, and
wherein said SEF allele encodes a polypeptide having at least about 90% amino acid sequence identity with SEQ ID NO:2 and comprises an arginine at a position corresponding to position 100 of SEQ ID NO:2, an alanine at a position corresponding to position 36 of SEQ ID NO:2, a glutamic acid at a position corresponding to position 248 of SEQ ID NO:2, a methionine at a position corresponding to position 255 of SEQ ID NO:2, a valine at a position corresponding to position 301 of SEQ ID NO:2, and/or an alanine at a position corresponding to position 608 of SEQ ID NO:2.
